(19) United States
(12) Patent Application Publication
(10) Pub. No.: US 2026/0144583 A1
(43) Pub. Date: May 28, 2026

(54) BONE FRACTURE REPAIR KIT, COMPONENTS, AND METHODS OF USE OF SAME

(71) Applicant: ARTHREX, INC., Naples, FL (US)

(72) Inventors: Alexander M. DelMonaco, Chelmsford, MA (US); Victoria N. Triaga, Naples, FL (US)

(21) Appl. No.: 19/402,568

(22) Filed: Nov. 26, 2025

Related U.S. Application Data
(60) Provisional application No. 63/725,935, filed on Nov. 27, 2024.
(60) Provisional application No. 63/810,651, filed on May 22, 2025.

(51) Int. Cl. A61B 17/88; A61B 17/00; A61B 17/72; A61B 17/90; A61B 90/00

(52) U.S. Cl. CPC A61B 17/8872; A61B 17/7291; A61B 17/90; A61B 90/03; A61B 2017/00477; A61B 17/7266; A61B 2090/034

(57) ABSTRACT

Disclosed herein is a fractured bone repair kit, components and methods of use of same. The kit includes a coupler that secures an intramedullary implant to a targeting guide that guides fasteners into bone fixation holes of the intramedullary implant to secure the intramedullary implant to the fractured bone. The coupler includes a sleeve and a fastener. The sleeve is releasably securable to the targeting guide such that the targeting guide is removable from the coupler and the intramedullary implant when attached to the coupler. An actuator of the targeting guide may be toggled to transition the kit from an assembled configuration in which the targeting guide is secured to the coupler to a released configuration in which the targeting guide is removable from the coupler.

### CROSS-REFERENCE TO RELATED APPLICATIONS

This application claims the benefit of Provisional Application No. 63/725,935, filed Nov. 27, 2024 and Provisional Application No. 63/810,651, filed May 22, 2025, which are hereby incorporated in their entireties herein.

### BACKGROUND

Bone fractures are a common medical condition both in the young and old segments of the population. One current treatment of bone fractures includes surgically resetting the fractured bone. After the surgical procedure, the fractured area of the body (e.g., a limb in which the fractured bone is located) is often placed in an external cast for an extended period of time to ensure that the fractured bone heals properly. This can take several months for the bone to heal and for the patient to remove the cast before resuming normal activities.

In some instances, an intramedullary (IM) nail is used to align and stabilize the fracture. In those instances, a metal rod is placed inside a canal of a bone and fixed in place, typically at both ends. Placement of conventional IM nails typically requires access collinear with a center line of the IM canal. For example, a fibula fracture may be treated by insertion of an IM nail. During insertion the IM nail enters a distal fragment of the fibula, is advanced across a fracture site, and enters a proximal fragment of the fibula. As used herein in reference to portions of anatomy (e.g., a bone such as a fibula), “proximal” refers to an object (e.g., a bone fragment) that is closer to the trunk or center mass of the body and “distal” refers to an object (e.g., a bone fragment that is farther from the trunk or center mass of the body.

Some known insertion and attachment systems for IM implants include an insertion and attachment guide that is not easily removable from the IM implant. This may result in the insertion and attachment guide only being removed once, when the IM implant is fully inserted and attached to the fractured bone. However, the inability to more easily remove and reattach the insertion and attachment guide to the implant throughout the procedure may hinder the implantation process.

### BRIEF SUMMARY

Some embodiments described herein provide a fracture repair system comprising a targeting guide that aligns fasteners used to attach an intramedullary implant to a fractured bone. The targeting guide is couplable to and decouplable from the intramedullary implant. Some embodiments of the targeting guide may be rapidly and repeatably couplable to and decouplable from the intramedullary implant. The fracture repair system may comprise a mechanism that is actuated (e.g., manipulated) to decouple the targeting guide from the intramedullary implant. In some embodiments of the fracture repair system described herein, the mechanism is positioned outside of the patient's body throughout the surgical procedure.

Injuries to the lower extremities frequently involve fibular fractures, which may occur in isolation or as part of bimalleolar or trimalleolar fracture patterns. Use of intramedullary implants/nails to repair a fractured fibula has gained popularity within the orthopedic community due to its advantages, including smaller incisions, reduced hardware irritation, lower rates of postoperative infection, and faster surgical times.

Traditionally, fibular nailing is performed via percutaneous insertion of the nail, followed by targeting of the distal interlocking holes using a targeting guide. However, a significant limitation of conventional targeting systems is the need to complete fixation of the lateral malleolus before addressing associated injuries, such as medial or posterior malleolar fractures. This constraint arises because the known targeting guides are typically rigidly attached to the intramedullary nail, which is often seated within the distal fibula. While detachment of some conventional targeting guides is possible, reattachment during surgery is cumbersome due to the use of thumb screws or captured bolts. Reattachment may also be difficult due to the attachment point between the targeting guide and the intramedullary nail (e.g., where the screw meets the nail) being inside the patient.

Furthermore, the presence of an external targeting guide can hinder intraoperative manipulation of the limb. Surgeons often need to invert or evert the limb with the fractured bone to assess fixation and perform stress testing, and some known external targeting guide obstruct these maneuvers. Additionally, manipulation of the limb with the targeting guide attached may result in interference from the surgical table. To prevent this interference, some surgical procedures elevate the limb above the surgical table, which can lead to additional complexity during the procedure (e.g., misalignment of the syndesmotic region).

Some embodiments of the fracture repair system described herein include a targeting guide system that addresses the limitations of conventional systems. For example, some embodiments of a targeting guide described herein include an latching mechanism that enables rapid attachment and detachment of the targeting guide from a nail attachment arm of the targeting guide. The structure of the nail attachment arm may relocate the coupling interface between the IM nail and the targeting guide to a position outside the patient's body, significantly improving ease of use.

Some disclosed embodiments of the targeting guide described herein may enhance surgical flexibility, reduce intraoperative complexity, and support a more comprehensive and efficient approach to managing complex ankle fractures. For example, the targeting guide as disclosed in some embodiments allows a surgeon to provisionally fix the lateral malleolus using an IM nail, temporarily detach the targeting guide from the IM nail to address medial and/or posterior malleolar fractures, and reattach the targeting guide as needed to finalize fixation. Additionally, a surgeon using the targeting guide as described herein may freely manipulate the limb with the fractured bone intraoperatively to perform stress testing and assessment of alignment, rotation, and length of the fractured bone as well as intraoperative assessment of the syndesmosis. Further, removal of the targeting guide described herein may provide ample working space to address ligamentous injuries, including the anterior inferior tibiofibular ligament (AITFL) and the deltoid ligament complex. Another added benefit is the option to use an open approach to perform a direct reduction of the syndesmosis, which may be more difficult or not possible due to interference of conventional guides along the open approach access path.

Some embodiments of a coupler described herein releasably attach an intramedullary implant to a targeting guide. The coupler secures the intramedullary implant to one or more fragments of a fractured bone via fasteners inserted through corresponding holes of the intramedullary implant and the targeting guide. The corresponding holes are aligned when the intramedullary implant is attached to the targeting guide by the coupler.

Some embodiments of the coupler comprise a sleeve and a fastener. The sleeve comprises a sleeve proximal end, a sleeve distal end, a sleeve body extending therebetween, and defining a sleeve lumen extending therethrough along a first longitudinal axis. The sleeve comprises an engagement portion that is shaped to releasably engage the targeting guide such that translation of the sleeve relative to the targeting guide along the first longitudinal axis is restricted. The fastener comprises a fastener proximal end, a fastener distal end, a fastener body extending therebetween, and defining a fastener lumen extending therethrough along a second longitudinal axis. The fastener distal end is shaped to engage the intramedullary implant such that translation of the fastener relative to the intramedullary implant along the second longitudinal axis is restricted. At least a portion of the fastener body is shaped to be inserted into and translated through the sleeve lumen.

Some embodiments of a coupler disclosed herein releasably attach an intramedullary implant to a targeting guide. The targeting guide is used to secure the intramedullary implant to one or more fragments of a fractured bone via fasteners. The fasteners are inserted through corresponding holes of the intramedullary implant and the targeting guide. The corresponding holes are aligned when the intramedullary implant is attached to the targeting guide by the coupler. The coupler comprises a sleeve and a fastener.

The sleeve comprises a sleeve proximal end, a sleeve distal end, a sleeve body extending therebetween, and defines a sleeve lumen extending therethrough along a first longitudinal axis. The sleeve comprises an engagement portion defining a recess that extends into the sleeve body radially toward the first longitudinal axis along a length of the engagement portion. The fastener comprises a fastener proximal end, a fastener distal end, a fastener body extending therebetween, and defines a fastener lumen extending therethrough along a second longitudinal axis. The fastener distal end comprises a threaded surface, and a cross-sectional shape of a distal portion of the fastener body fits within the sleeve lumen.

Some embodiments of a bone fracture repair kit comprise an intramedullary implant having a bone fixation hole that receives a fastener to secure the intramedullary implant to a bone. The kit further comprises a targeting guide and a coupler. The bone fracture repair kit defines an assembled configuration in which an actuator of the targeting guide engages an engagement portion of the sleeve thereby securing the intramedullary implant to the targeting guide such that movement of the intramedullary implant relative to the targeting guide is restricted. In the assembled configuration the bone fixation hole is aligned with a fastener guide hole of the targeting guide. The bone fracture repair kit further defines a released configuration in which the actuator is disengaged from the engagement portion and the targeting guide is movable relative to the coupler.

Some embodiments of the targeting guide described herein include components that are removable/reversible. For example, a portion of the targeting guide may be attached in a first configuration to secure an intramedullary implant within a bone in a left limb (e.g., a left fibula). The portion may be removable from the targeting guide and reattached in a second configuration to secure an intramedullary implant within a bone in a right limb (e.g., a right fibula).

Some embodiments of a method of assembling a bone fracture repair kit are described herein. The method comprises assembling a coupler comprising a fastener and a sleeve. The sleeve has sleeve proximal end, a sleeve distal end, a sleeve body extending therebetween, and a sleeve lumen extending therethrough along a first longitudinal axis. The fastener has a fastener proximal end, a fastener distal end, a fastener body extending therebetween, and defines a fastener lumen extending therethrough along a second longitudinal axis.

The method comprises inserting the fastener distal end into and through the sleeve lumen. The method further comprises connecting the fastener distal end to an intramedullary implant while a portion of the fastener is within the sleeve lumen. According to the method an actuator of a targeting guide abuts an engagement portion of the sleeve to secure a position of the sleeve relative to the targeting guide. The method further comprises disengaging the actuator from the engagement portion of the sleeve. The targeting guide is moved relative to the sleeve, fastener, and intramedullary implant while the actuator is disengaged from the engagement portion.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not necessarily drawn to scale, and some of these elements may be arbitrarily enlarged and positioned to improve drawing legibility. Further, the particular shapes of the elements as drawn, are not necessarily intended to convey any information regarding the actual shape of the particular elements, and may have been solely selected for ease of recognition in the drawings. The headings and Abstract of the Disclosure provided herein are for convenience only and do not interpret the scope or meaning of the embodiments.

FIG. 1 is an anterior view of a fractured fibula.

FIG. 2 is an anterior view of the fractured fibula illustrated in FIG. 1 being prepared to receive an intramedullary implant.

FIG. 3 is an anterior view of the fractured fibula illustrated in FIG. 2 and a bone fracture repair kit in use to repair the fractured fibula.

FIG. 4 is a side, rear isometric view of the bone fracture repair kit illustrated in FIG. 3, the bone fracture repair kit including an intramedullary implant, a targeting guide, and a coupler in an assembled configuration.

FIG. 5 is a side elevation view of the bone fracture repair kit illustrated in FIG. 4 in the assembled configuration.

FIG. 6 is a front elevation view of the bone fracture repair kit illustrated in FIG. 4 in the assembled configuration.

FIG. 7 is a side, rear, isometric, exploded view of the bone fracture repair kit illustrated in FIG. 4.

FIG. 8 is a front elevation view of a sleeve of the coupler illustrated in FIG. 4.

FIG. 9 is a cross-sectional view of the sleeve illustrated in FIG. 8.

FIG. 10 is a bottom plan view of the sleeve illustrated in FIG. 8.

FIG. 11 is a front elevation view of a fastener of the coupler illustrated in FIG. 7.

FIG. 12 is a cross-sectional view of the fastener illustrated in FIG. 11.

FIG. 13 is a rear elevation view of the targeting guide illustrated in FIG. 4.

FIG. 14 is a side, top, front isometric view of the targeting guide illustrated in FIG. 13.

FIG. 15 is a cross-sectional view of the targeting guide illustrated in FIG. 14.

FIG. 16 is a cross-sectional view of the bone fracture repair kit illustrated in FIG. 4 in the assembled configuration.

FIG. 17 is a cross-sectional view of the bone fracture repair kit illustrated in FIG. 16 in a released configuration.

FIG. 18 is a top, first side isometric view of the intramedullary implant illustrated in FIG. 4.

FIG. 19 is a bottom, second side isometric view of the intramedullary implant illustrated in FIG. 18.

FIG. 20 is a cross-sectional view of the intramedullary implant illustrated in FIG. 18 in a first orientation with a deployable fixator in an insertion configuration.

FIG. 21 is a cross-sectional view of the intramedullary implant illustrated in FIG. 20 in the first orientation with the deployable fixator in a deployed configuration.

FIG. 22 is a cross-sectional view of the intramedullary implant illustrated in FIG. 18 in a second orientation with the deployable fixator in an insertion configuration.

FIG. 23 is a cross-sectional view of the intramedullary implant illustrated in FIG. 22 in a third orientation with the deployable fixator in an insertion configuration.

FIG. 24 is a bottom plan view of the intramedullary implant illustrated in FIG. 18.

FIG. 25 is a top, first side isometric view of another intramedullary implant.

FIG. 26 is a posterior-lateral view of the bone fracture repair kit in use to repair the fractured fibula illustrated in FIG. 3 with the bone fracture repair kit in the assembled configuration.

FIG. 27 is a posterior-lateral view of the bone fracture repair kit in use to repair the fractured fibula illustrated in FIG. 26 with the bone fracture repair kit in the released configuration.

FIG. 28 is a posterior-lateral view of the bone fracture repair kit in use to repair the fractured fibula illustrated in FIG. 26, with temporary bone fixators inserted through a multi-lumen cannula into a first fragment of the fractured fibula.

FIG. 29 is an anterior view of the fractured fibula, bone fracture repair kit, multi-lumen cannula, and temporary bone fixators illustrated in FIG. 28, with a force being applied to the first fragment of the fractured bone.

FIG. 30 is a medial view of the fractured fibula, bone fracture repair kit, and temporary bone fixators illustrated in FIG. 28.

FIG. 31 is an anterior view of the fractured fibula, bone fracture repair kit, multi-lumen cannula, and temporary bone fixators illustrated in FIG. 28, with an actuator deploying internal fixators of the intramedullary implant.

FIG. 32 is an anterior view of the fractured fibula, bone fracture repair kit, multi-lumen cannula, and temporary bone fixators illustrated in FIG. 28, with a permanent bone fixator securing the intramedullary implant to the first fragment of the fractured bone and a second permanent bone fixator securing the intramedullary implant to the second fragment of the fractured bone.

FIG. 33 is an anterior view of the fractured fibula and intramedullary implant illustrated in FIG. 32, with permanent bone fixators securing the intramedullary implant to the first fragment of the fractured bone.

FIG. 34 is an anterior view of the fractured fibula and intramedullary implant illustrated in FIG. 33, with permanent bone fixators securing the intramedullary implant to the first and second fragments of the fractured bone and an end cap being secured to the intramedullary implant.

FIG. 35 is an anterior view of the intramedullary implant illustrated in FIG. 34 secured within the fractured fibula upon completion of a surgical procedure to repair the fractured fibula.

### DETAILED DESCRIPTION

As noted above, conventional bone fracture repair kits that include an intramedullary implant (e.g., a fibular nail) are typically attached to an insertion device (e.g., a targeting guide) during insertion into the intramedullary canal and fixation to the fractured bone. While attached to the insertion device, the position and orientation of the intramedullary implant may be adjusted within the intramedullary canal of the fractured bone. Once fully inserted and secured to the fractured bone, the insertion device is detached from the intramedullary implant to complete the procedure.

However, there may be advantages to an insertion device and targeting guide that is removable from and able to be reattached to an intramedullary implant during a bone fracture repair procedure. For example, a significant limitation of conventional repair kits for fractured fibulas is the need to complete fixation of the lateral malleolus before addressing associated injuries, such as medial or posterior malleolar fractures. This constraint arises in known targeting guides that are rigidly attached to the intramedullary nail, which is often seated within the distal fibula during the repair procedure. Furthermore, the presence of an external targeting guide attached to an implanted intramedullary implant can hinder intraoperative manipulation of the limb. Surgeons often need to invert or evert the limb with the fractured bone to assess fixation and perform stress testing, and some known external targeting guides obstruct these maneuvers.

Advantageously, a coupler as described herein enables quick release and removal of the targeting guide from the intramedullary implant, and easy reattachment to the intramedullary implant during a surgical procedure. Thus, an intramedullary implant may be attached and secured relative to the targeting guide during insertion into an intramedullary canal. Then, the targeting guide may be disengaged from the intramedullary implant (via the coupler) before final fixation of the intramedullary implant to the fractured bone. After performing one or more actions (e.g., treatment of other injuries, manipulation of the limb, etc.), the targeting guide may be reattached to the intramedullary implant (e.g., via the coupler).

With the targeting guide attached to the intramedullary implant, fasteners may be inserted (e.g., guided by the targeting guide) to secure the intramedullary implant to the fractured bone. The targeting guide may then be removed/detached from the intramedullary implant to complete the procedure. According to some embodiments, the coupler facilitates quick removal of the targeting guide (e.g., via a push button or actuation of a spring biased lever without the use of a screw or captured bolt).

Some embodiments of the coupler described herein may be included as part of a kit. The kit may comprise one or more of intramedullary implants (e.g., in various sizes, lengths, widths, etc.), a targeting guide that assists in placement of temporary and/or permanent fixators (e.g., k-wires, bone screws, etc.) to secure the intramedullary implant relative to a fractured bone, a coupler, an actuator, drills, reamers, or any combination thereof.

The intramedullary implant may comprise a deployable fixator (e.g., one or more fingers or talons) that are inserted into an intramedullary canal (e.g., along a length of the intramedullary canal) in an insertion configuration having a narrow profile. Once positioned within an IM cavity (e.g., after crossing a fracture site), the deployable fixator may be “deployed” (e.g., transitioned from the insertion configuration to a deployed configuration). In the deployed configuration the deployable fixator may have a wider profile. For example, in the insertion configuration the deployable fixator may be movable within the IM cavity of the fractured bone and in the deployed configuration the deployable fixator may abut the inner wall of the IM cavity of the fractured bone, thereby resisting movement of the IM nail relative to the fractured bone.

Conventional IM nails that rely solely on a deployable fixator positioned within the IM cavity for fixation relative to one or more of the fragments of a fractured bone may experience some amount of movement or slippage of the conventional IM nail relative to the bone fragment. This movement during fixation of the IM nail, or during the healing process may negatively impact the final result of the treatment of the fractured bone. Advantageously, some embodiments of the coupler disclosed herein define a path for an actuator that deploys the deployable fixator.

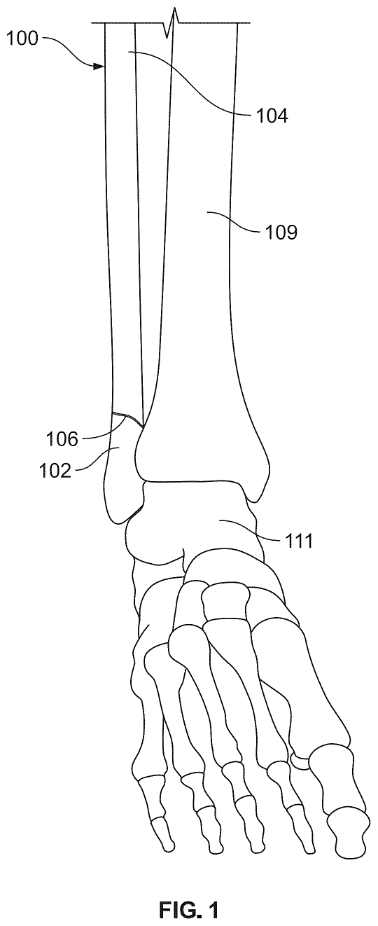

Referring now to the drawings, and specifically to FIGS. 1 to 3, a fractured bone 100 may comprise two or more fragments (e.g., a first bone fragment 102 and a second bone fragment 104) separated by one or more fractures 106. For example, the fractured bone 100 may be a fibula, the first bone fragment 102 may be a distal fragment of the fibula, and the second bone fragment 104 may be a proximal fragment of the fibula. As shown in FIG. 1, prior to insertion of any fixation hardware, the first bone fragment 102 and the second bone fragment 104 may be aligned (e.g., to approximate the shape of the fractured bone 100 prior to the fracture 106—referred to herein as the “pre-fracture condition”). A clamp (not shown) may be used to hold the first bone fragment 102, the second bone fragment 104, or both in place.

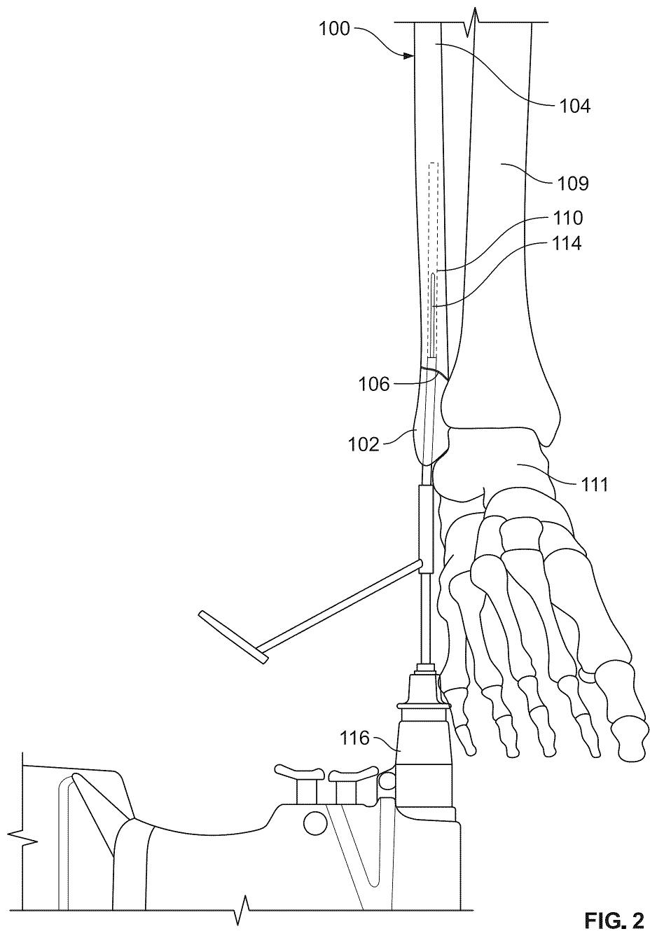

As shown in FIG. 2, instruments may be used to form a hole 108 within the fractured bone 100. The hole 108 may include an intramedullary canal 110 of the fractured bone 100. The intramedullary canal 110 may be enlarged along at least a portion of the length of the fractured bone 100 to a size that can accommodate an intramedullary implant. For example, a guide wire 114 may be inserted through a surface in the first bone fragment 102 (e.g., a distal surface of the lateral malleolus) and into the intramedullary canal 110. The guide wire 114 may be advanced (e.g., proximally/anteriorly) across the fracture 106 into the intramedullary canal 110 of the second bone fragment 104. A drill or reamer 116 may be used to form the hole 108 (e.g., by enlarging the intramedullary canal 110 to a desired size). The drill or reamer 116 may be cannulated and inserted over the guide wire 114 and advanced along the guide wire 114 through the first bone fragment 102, across the fracture 106, and into the second bone fragment 104.

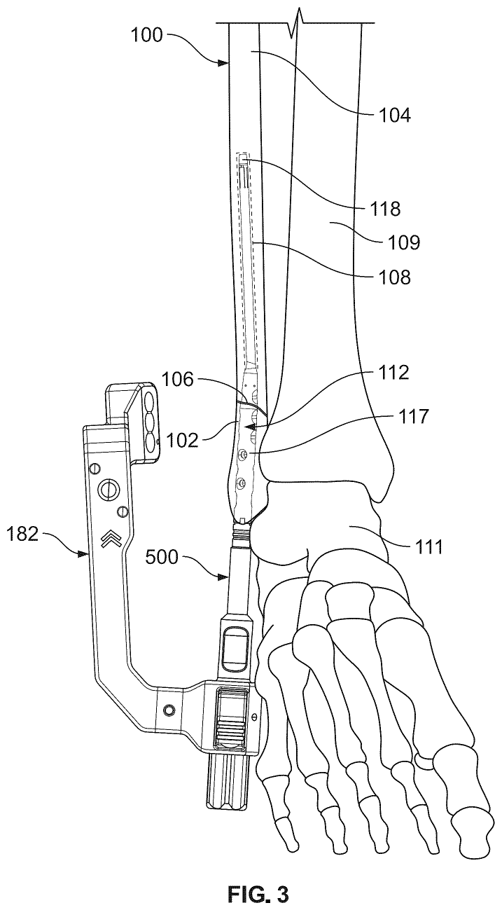

Once the hole 108 is formed, an intramedullary implant 112 (e.g., an intramedullary nail 117) may be positioned within the hole 108, for example as shown in FIG. 3. A tip 118 of the intramedullary nail 117 may be connected to a targeting guide 182 via a coupler 500 and advanced (e.g., proximally/anteriorly) through a surface in the first bone fragment 102 (e.g., the distal surface of the lateral malleolus), across the fracture 106, and into the second bone fragment 104. Once positioned in the hole 108 as desired, the intramedullary nail 117 may be secured to both the first bone fragment 102 and the second bone fragment 104 of the fractured bone 100, as described in further detail below.

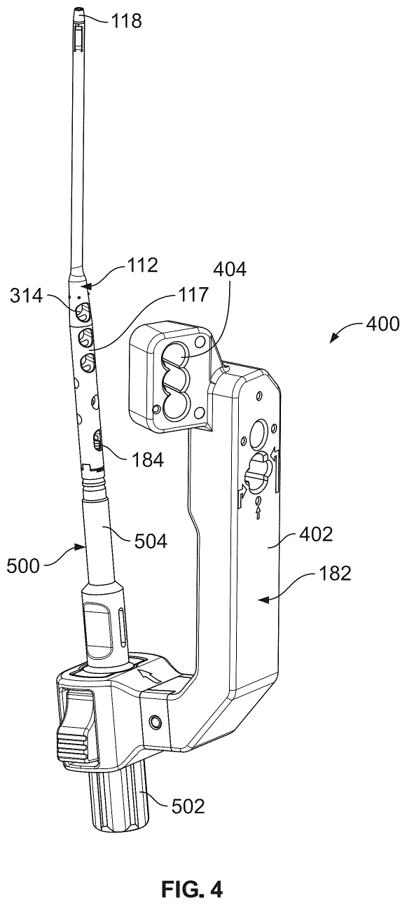

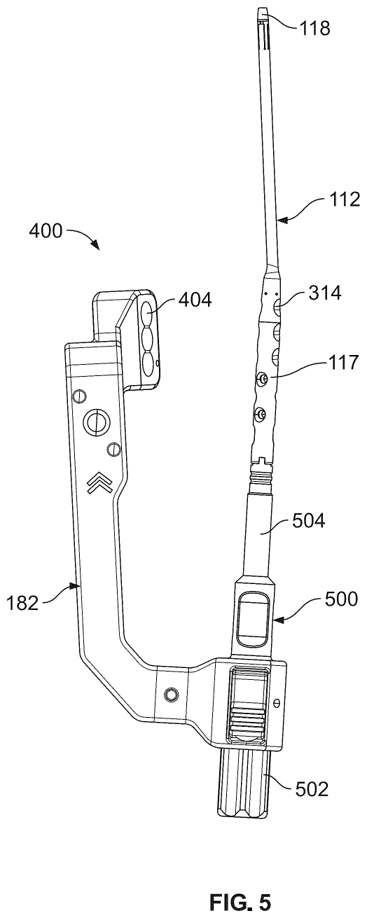

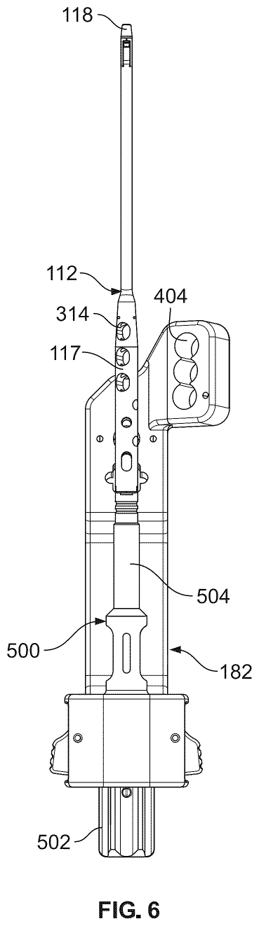

Referring to FIGS. 4 to 7, the targeting guide 182 may be used to insert and secure the intramedullary implant 112 to the fractured bone 100. As shown, the targeting guide 182 may comprise a guide body 402 that defines one or more guide holes 404. The targeting guide 182 may be connected to the intramedullary implant 112 (e.g., the intramedullary nail 117) during an insertion/implantation procedure. As described in further detail below, when connected to the intramedullary nail 117 one or more of the guide holes 404 may be aligned with a corresponding transverse fixation hole 314 of the intramedullary nail 117. When aligned, a fastener (e.g., a temporary fastener such as a k-wire and/or a permanent fastener such as a bone screw) may be inserted through the guide hole 404 and then into the corresponding transverse fixation hole 314.

Some embodiments of a bone fracture repair kit 400 may comprise one or more intramedullary implants 112 (e.g., the intramedullary nail 117 or multiple intramedullary nails 117 of different sizes/lengths), the targeting guide 182, and one or more couplers 500 that releasably connect one of the intramedullary implants 112 to the targeting guide 182. As described in further detail below, the coupler 500 may connect the intramedullary nail 117 to the targeting guide 182 such that the guide hole(s) 404 are aligned with a corresponding transverse fixation hole 314. The coupler 500 may connect the intramedullary nail 117 to the targeting guide 182 such that translation and/or rotation of the intramedullary nail 117 relative to the targeting guide 182 is restricted (e.g., prevented). When connected to the targeting guide 182 via the coupler 500, the position and orientation of the intramedullary nail 117 within the hole 108 may be manipulated by moving/manipulating the targeting guide 182.

Some embodiments of the coupler 500 may be releasably coupled to the intramedullary nail 117. For example, prior to insertion of the intramedullary nail 117 into the hole 108, a fastener 502 of the coupler 500 may be coupled to the intramedullary nail 117 and a sleeve 504 of the coupler 500 may be coupled to the targeting guide 182. Thus, the intramedullary nail 117 may be coupled to the targeting guide 182 via the coupler 500 (e.g., a connection between the fastener 502 and the sleeve 504). The bone fracture repair kit 400 may comprise an assembled configuration in which the intramedullary nail 117 and the targeting guide 182 are connected via the coupler 500. When in the assembled configuration, the bone fracture repair kit 400 may be used to position the intramedullary nail 117 within a fractured bone.

According to some embodiments, the coupler 500 may be selectively releasable from the intramedullary nail 117, the targeting guide 182, or both. For example, the fastener 502 may be decoupled from the intramedullary nail 117 while the sleeve 504 remains coupled to the targeting guide 182. In such a configuration, the targeting guide 182 and the coupler 500 may be moved (e.g., simultaneously) relative to the intramedullary nail 117. Such removal may be beneficial after the intramedullary nail 117 is secured to a fractured bone prior to the conclusion of a surgical procedure.

Additionally, the sleeve 504 may be selectively decoupled from the targeting guide 182 while the fastener 502 remains coupled to the intramedullary nail 117. In such a configuration, the targeting guide 182 may be moved relative to both the intramedullary nail 117 and the coupler 500, which is attached thereto. Such removal may be beneficial after initial placement of the intramedullary nail 117 within a fractured bone, but prior to final fixation of the intramedullary nail 117 to the fractured bone. Removal of the targeting guide 182 may make it easier to perform one or more actions (e.g., treatment of other injuries, manipulation of the limb in which the intramedullary nail 117 is being inserted, etc.). The targeting guide 182 may then be recoupled to the sleeve 504 to once again couple the targeting guide 182 to the intramedullary nail 117 such that the guide holes 404 are aligned with the transverse fixation holes 314.

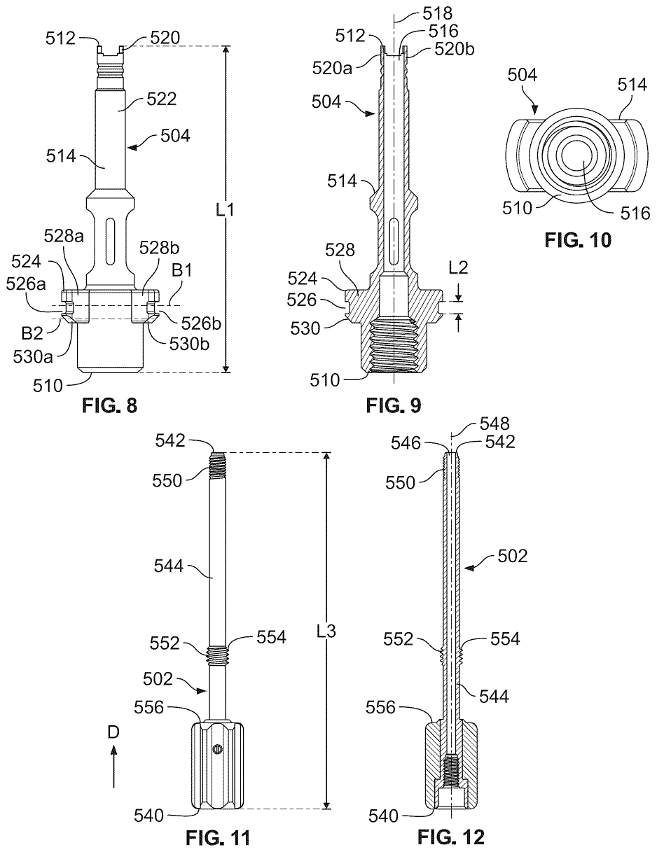

Referring to FIGS. 8 to 10, some embodiments of the sleeve 504 may comprise a sleeve proximal end 510, a sleeve distal end 512, and a sleeve body 514 extending therebetween. The sleeve 504 may define a sleeve lumen 516 extending through the sleeve body 514 (e.g., along a first longitudinal axis 518 through a length L1 of the sleeve body 514).

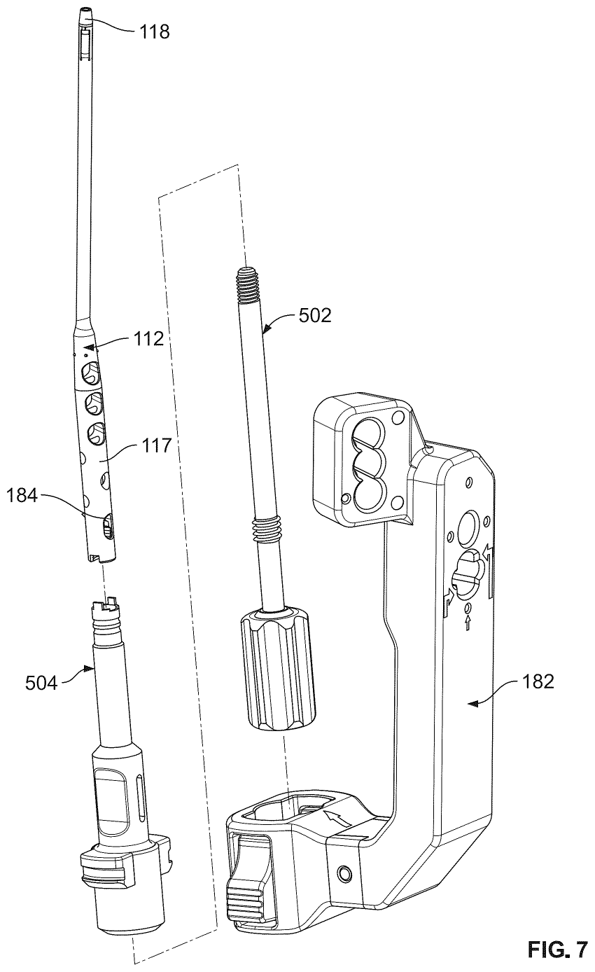

The sleeve distal end 512 may be shaped to engage an intramedullary implant (e.g., the intramedullary nail 117 shown in FIG. 7) such that rotation of the sleeve 504 relative to the intramedullary implant about the first longitudinal axis 518 is restricted. For example, some embodiments of the sleeve distal end 512 may comprise a flange 520 extending distally from a remainder 522 of the sleeve body 514. As shown, the flange 520 may define a radial length that is less than a full circumference around the first longitudinal axis 518. For example, the radial length may range from 10 to 90 degrees. Some embodiments of the sleeve 504 may comprise multiple flanges 520 (e.g., a first flange 520a and a second flange 520b) each extending distally from the remainder 522 of the sleeve body 514. The radial length of each of the flanges 520 may be equal to one another, or may vary between adjacent ones of the flanges 520.

As described in detail below, the sleeve 504 may comprise an engagement portion 524 that is shaped to releasably engage the targeting guide 182 such that translation of the sleeve 504 relative to the targeting guide 182 along the first longitudinal axis 518 is restricted. For example, the engagement portion 524 of the sleeve 504 may define a recess 526 that extends into the sleeve body 514 (e.g., radially toward the first longitudinal axis 518). The recess 526 may be in the form of a notch with a length L2 measured parallel to the first longitudinal axis 518.

According to some embodiments, the sleeve body 514 may comprise a projection 528 that extends radially outward (e.g., away from the first longitudinal axis 518) along a portion of an outer perimeter of the sleeve body 514. The recess 526 may extend into the projection 528 as shown in the illustrated embodiment. The engagement portion 524 may comprise a tapered surface 530 that extends radially away from the first longitudinal axis 518 as the engagement portion 524 extends distally (e.g., toward the sleeve distal end 512). As shown, the tapered surface 530 may be positioned proximally of the recess 526 such that the tapered surface 530 functions as a ramp along which a surface of the targeting guide 182 travels before entering the recess 526. The ramp may enable transition of the bone fracture repair kit 400 from the released configuration to the assembled configuration without actuating any actuators (e.g., pushing any buttons/levers).

Some embodiments of the engagement portion 524 may comprise multiple recesses 526. For example, the engagement portion 524 may comprise a first tapered surface 530a and a first recess 526a that form part of/extend into a first projection 528a, and the engagement portion may further comprise a second tapered surface 530b and a second recess 526b that form part of/extend into a second projection 528b. As shown in the illustrated embodiment, the first recess 526a and the second recess 526b may be positioned such that a first plane B1 that is normal to the first longitudinal axis 518 intersects both the first recess 526a and the second recess 526b. Similarly, the first tapered surface 530a and the second tapered surface 530b may be positioned such that a second plane B2 that is normal to the first longitudinal axis 518 intersects both the first tapered surface 530a and the second tapered surface 530b.

Referring to FIGS. 11 and 12, some embodiments of the fastener 502 may comprise a fastener proximal end 540, a fastener distal end 542, and a fastener body 544 extending therebetween. The fastener 502 may define a fastener lumen 546 extending through the fastener body 544 (e.g., along a second longitudinal axis 548 through a length L3 of the fastener body 544).

The fastener distal end 542 may be shaped to engage an intramedullary implant (e.g., the intramedullary nail 117 shown in FIG. 7) such that translation of the fastener 502 relative to the intramedullary implant about the second longitudinal axis 548 is restricted. For example, some embodiments of the fastener distal end 542 may comprise threads 550 (e.g., external threads) that threadedly engage with corresponding threads of the intramedullary implant. As shown, the threads 550 may extend along a portion of the length L3 of the fastener 502. For example, the threads 550 may extend along less than half (e.g., less than a quarter) of the length L3.

As will be described in greater detail below, at least a portion of the fastener body 544 may be insertable into and translatable through the sleeve lumen 516. For example, the fastener 502 may be positionable within the sleeve lumen 516 such that at least a portion of the threads 550 of the fastener distal end 542 extends distally beyond sleeve distal end 512 (e.g., when the bone fracture repair kit 400 is in the assembled configuration).

Some embodiments of the fastener 502 may comprise an engagement portion 552 that is shaped to engage the sleeve 504 to restrict translation of the fastener 502 relative to the sleeve 504 along the first longitudinal axis 518 and/or the second longitudinal axis 548. The engagement portion 552 may comprise a threaded surface (e.g., external threads 554 as shown in the illustrated embodiment) that engages with a corresponding surface (e.g., internal threads 532 within the sleeve lumen 516 shown in FIG. 9) of the sleeve 504.

Some embodiments of the fastener 502 may comprise a stop 556 that limits translation of the fastener 502 relative to the sleeve 504 (e.g., in a distal direction D). The stop 556 may be in the form of a shoulder with a cross-sectional diameter that is larger than a cross-sectional diameter of the sleeve lumen 516. Thus, in the assembled configuration the stop 556 may abut the sleeve proximal end 510 and remain positioned outside the sleeve lumen 516.

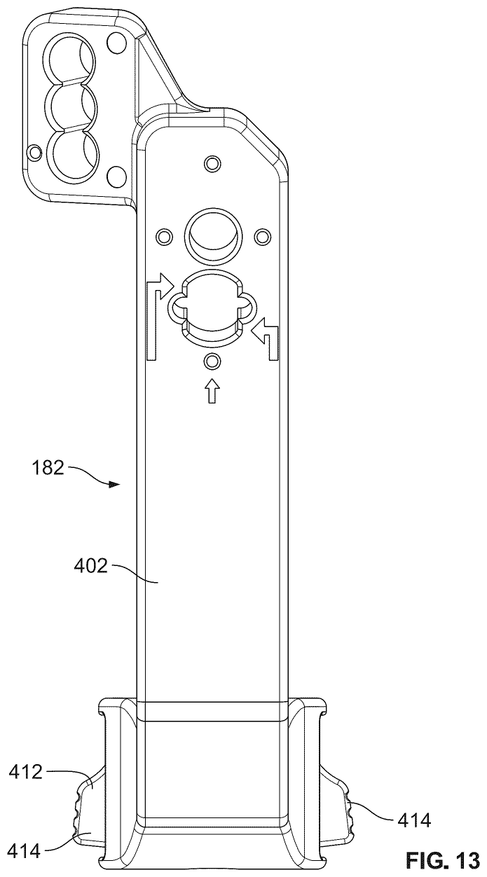

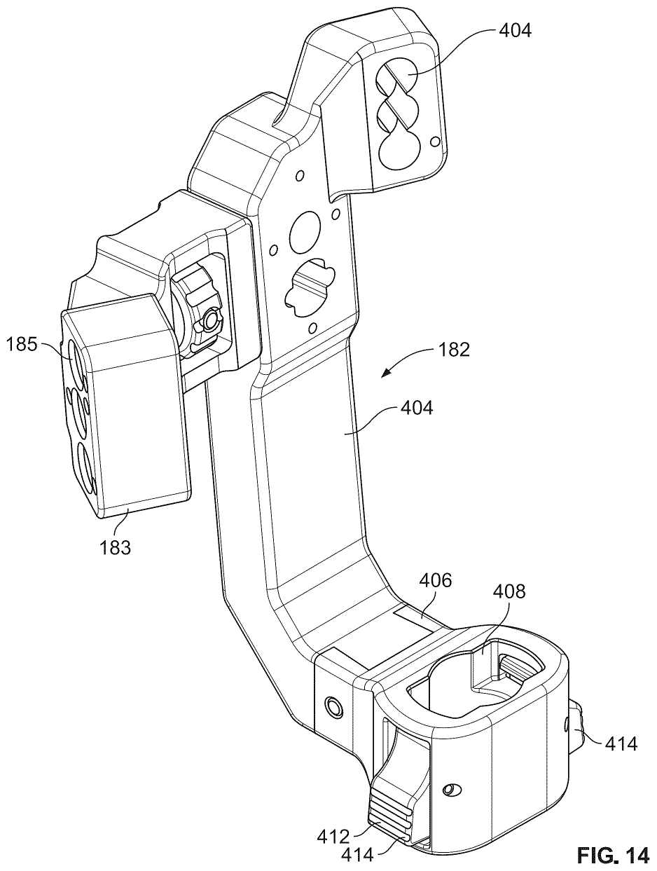

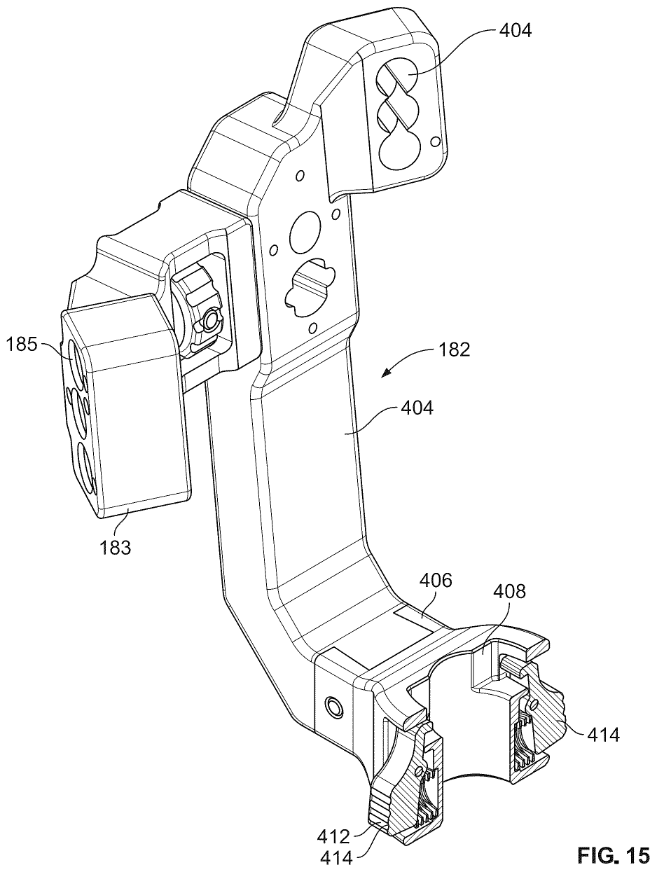

Referring to FIGS. 13 to 15, the guide body 402 of the targeting guide 182 may comprise a boom arm 406 that defines a through hole 408 that is sized to receive the coupler 500 therein. The through hole 408 may extend through the boom arm 406 along a direction that is offset from the one or more guide holes 404. According to some embodiments, the through hole 408 may have a non-circular cross-section that corresponds to an outer perimeter of the coupler 500 (e.g., the sleeve 504) such that when the coupler is positioned within the through hole 408 rotation of the coupler 500 relative to the targeting guide 182 is restricted (e.g., prevented).

The targeting guide 182 may comprise a locking mechanism 412 that secures the targeting guide 182 to the coupler 500 in the assembled configuration. For example, the locking mechanism 412 may comprise an actuator 414. The actuator may be biased (e.g., spring biased) to a locked configuration, and may be actuated (e.g., by exertion of a force on the actuator 414) into an unlocked configuration. The locking mechanism 412 may comprise multiple actuators 414. As shown the targeting guide 182 may include a plurality of actuators 414 (e.g., a number of actuators 414 that matches the number of recesses 526 of the sleeve 504).

Some embodiments of the targeting guide 182 may comprise a removable and reversible component 183 that is detachable from a remainder of the targeting guide 182. The removable and reversible component 183 may include a number of guide holes 185 that align with corresponding bone fixation holes of an intramedullary implant. The guide holes 185 may be arranged in the removable and reversible component 183 such that when attached to a first targeting guide in a first orientation, the guide holes 185 are aligned with an intramedullary implant to be implanted within a left fibula. The guide holes 185 may further be arranged such that when the removable and reversible component 183 is attached to a second targeting guide in a second orientation (e.g., flipped vertically), the guide holes 185 are aligned with an intramedullary implant to be implanted within a right fibula.

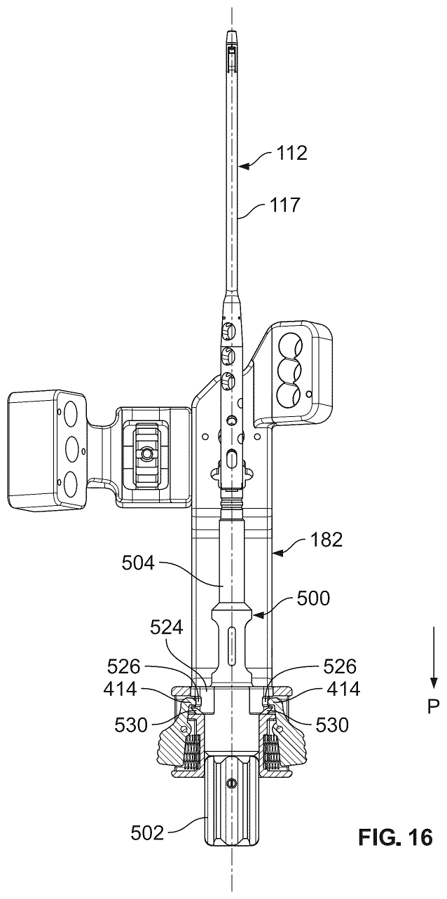

Referring to FIGS. 13 to 17, the coupler 500 may be selectively secured to and released from the targeting guide 182. As shown in FIG. 16, the engagement portion 524 of the sleeve 504 may be positioned within the through hole 408 of the targeting guide 182. According to some embodiments, the fastener proximal end 540 may be passed through the through hole 408 and advanced in a proximal direction P until the actuator(s) 414 is/are secured within the recess(es) 526. A portion of the actuator 414 may ride along the tapered surface 530 and may then be biased to enter the recess 526. Upon entering the recess 526, the actuator 414 may be trapped therein restricting (e.g., preventing) movement of the coupler 500 relative to the targeting guide 182.

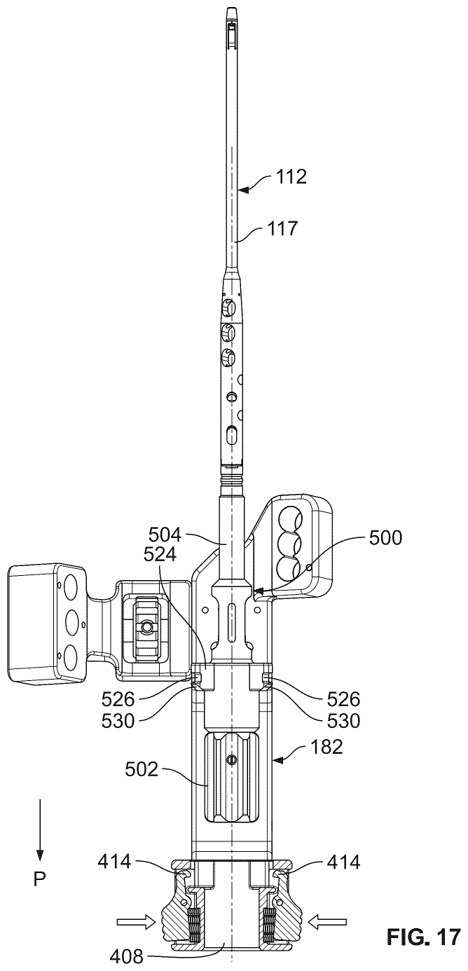

As shown in FIG. 17, the targeting guide 182 may be disengaged from the coupler 500 in a released configuration of the bone fracture repair kit 400. According to some embodiments, a force may be applied to the actuator 414 (e.g., a lever or button) to remove the actuator 414 from the recess 526. With the actuator 414 removed, the targeting guide 182 is movable (e.g., translatable) relative to the coupler 500. For example, the targeting guide 182 may be moved in the proximal direction P relative to the coupler 500 until an entirety of the coupler 500 is removed from the through hole 408.

Prior to (or after) securing the coupler 500 to the targeting guide 182, the coupler 500 may be selectively secured to the intramedullary nail 117. According to some embodiments, the fastener 502 may be rotated relative to the intramedullary nail 117 (and relative to the sleeve 504) about the second longitudinal axis 548. Rotating the fastener 502 relative to the intramedullary nail 117 with the threads 550 engaged with corresponding internal threads of the intramedullary nail 117 results in threaded engagement of the coupler 500 and the intramedullary nail 117. As the fastener 502 rotates it translates relative to the intramedullary nail 117 (e.g., until the stop 556 abuts the sleeve 504 and/or until the corresponding threads bottom out).

Thus, according to some embodiments of the bone fracture repair kit 400, the sleeve 504 may be captured between the fastener 502 (e.g., the stop 556) and the intramedullary nail 117. Exerting a rotational force on the fastener 502 (e.g., the stop 556) may rotate the fastener 502 relative to the intramedullary nail 117 to disengage the threads 550 and release the fastener 502 from the intramedullary nail 117.

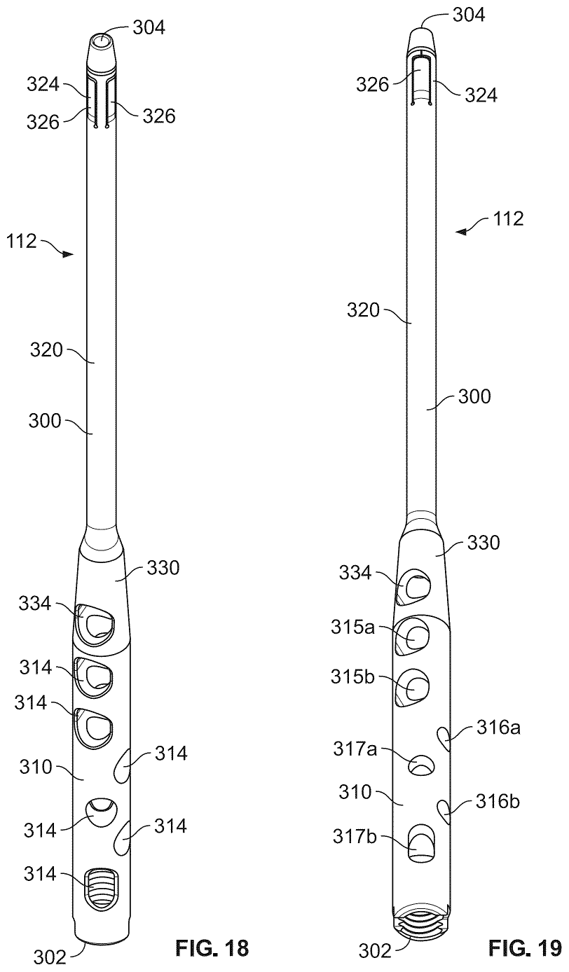

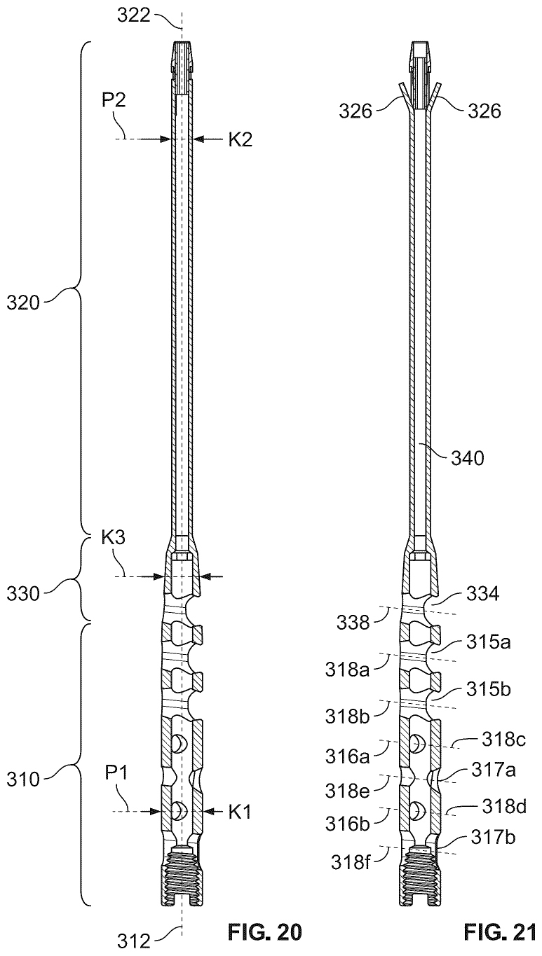

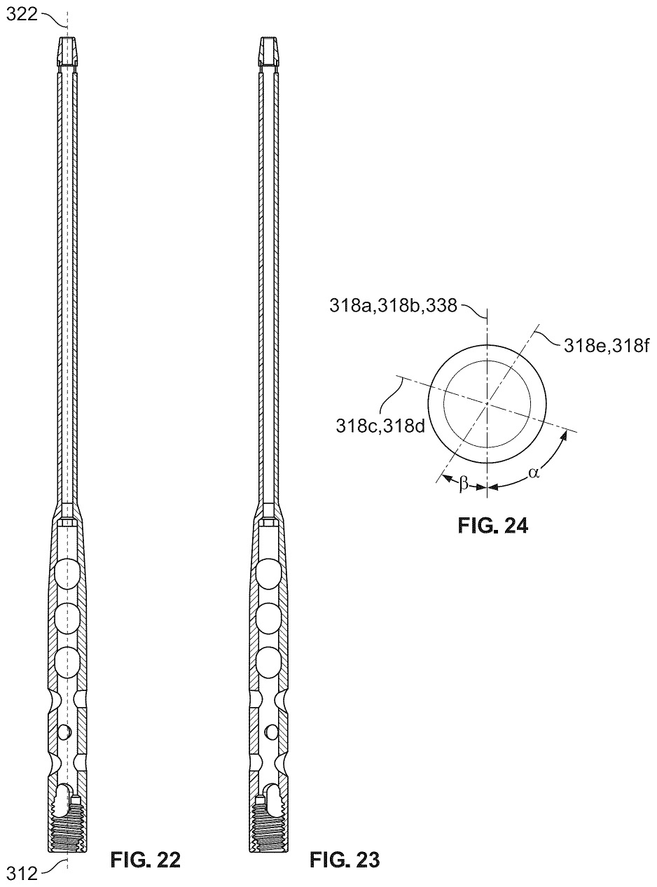

Referring to FIGS. 18 to 25, some embodiments of the intramedullary implant 112 described herein comprise an implant body 300 that extends from a proximal end 302 to a distal end 304. The implant body 300 may comprise a proximal body portion 310 that extends along a first central axis 312. The proximal body portion 310 may define a plurality of transverse fixation holes 314 that each extend through the proximal body portion 310 and intersect the first central axis 312. As shown, the proximal body portion 310 may define a first maximum cross-sectional dimension K1 measured in a first plane P1 that is normal to the first central axis 312. The proximal body portion 310 may be cylindrical or another shape with a constant cross-sectional dimension K1 along at least a portion of a length of the proximal body portion 310. According to some embodiments, one or more portions up to an entirety of the proximal body portion 310 may be tapered (e.g., having a maximum cross-sectional dimension that decreases distally).

The plurality of transverse fixation holes 314 may comprise multiple subsets of transverse fixation holes (e.g., a first subset of transverse fixation holes 315, a second subset of transverse fixation holes 316, and a third subset of transverse fixation holes 317). Each of the multiple subsets may comprise a single transverse fixation hole 314, or may comprise multiple (e.g., two or more, for example between two and five) transverse fixation holes 314. As shown, each of the plurality of transverse fixation holes 314 within a given subset may be parallel to one another and angularly offset from those of the plurality of transverse fixation holes 314 within a different subset.

For example, the first subset of transverse fixation holes 315 may comprise a distal-most one of the plurality of transverse fixation holes 314. According to some embodiments, the first subset of transverse fixation holes 315 may comprise a first transverse fixation hole 315a (e.g., the distal-most one of the plurality of transverse fixation holes 314) and a second transverse fixation hole 315b, positioned proximally of the first transverse fixation hole 315a.

The second subset of transverse fixation holes 316 may comprise at least one of the plurality of transverse fixation holes 314 positioned proximally of the first transverse fixation hole 315a. As shown, the second subset of transverse fixation holes 316 may comprise a third transverse fixation hole 316a and a fourth transverse fixation hole 316b, positioned proximally of the third transverse fixation hole 316a. According to some embodiments, the third transverse fixation hole 316a and the fourth transverse fixation hole 316b may both be positioned proximally of both the first transverse fixation hole 315a and the second transverse fixation hole 315b.

The third subset of transverse fixation holes 317 may comprise at least one of the plurality of transverse fixation holes 314 positioned proximally of the third transverse fixation hole 316a. As shown, the third subset of transverse fixation holes 317 may comprise a fifth transverse fixation hole 317a and a sixth transverse fixation hole 317b, positioned proximally of the fifth transverse fixation hole 317a. According to some embodiments, the second subset of transverse fixation holes 316 and the third subset of transverse fixation holes 317 may be arranged alternatingly. For example, the plurality of transverse fixation holes 314 may be arranged distal to proximal as follows: the first transverse fixation hole 315a, the second transverse fixation hole 315b, the third transverse fixation hole 316a, the fifth transverse fixation hole 317a, the fourth transverse fixation hole 316b, and the sixth transverse fixation hole 317b.

Some embodiments of the intramedullary implant 112 may comprise a distal body portion 320 that extends along a second central axis 322 that is either aligned with or angularly offset from the first central axis 312. As shown, the first central axis 312 and the second central axis 322 may be angularly offset within a range of 0 degrees to 25 degrees, e.g., from 2 degrees to 20 degrees, from 3 degrees to 15 degrees, from 4 degrees to 10 degrees, or from 5 degrees to 8 degrees. In terms of lower limits, the first central axis 312 and the second central axis 322 may be angularly offset by greater than 2 degrees, e.g., greater than 3 degrees, greater than 4 degrees, greater than 5 degrees, greater than 6 degrees, greater than 8 degrees, greater than 10 degrees, greater than 15 degrees, or greater than 20 degrees. In terms of upper limits, the first central axis 312 and the second central axis 322 may be angularly offset by less than 25 degrees, e.g., less than 20 degrees, less than 15 degrees, less than 10 degrees, less than 8 degrees, less than 6 degrees, less than 5 degrees, less than 4 degrees, or less than 3 degrees. For example, the first central axis 312 and the second central axis 322 may be angularly offset by about 6 degrees (e.g., plus or minus 2 degrees). The distal body portion 320 may comprise a deployable fixation element 324 that transitions from an insertion configuration to a deployed configuration. For example, the deployable fixation element 324 may include one or more movable talons 326 or fingers that transition from an insertion configuration with a narrower profile to a deployed configuration with a wider profile.

In the insertion configuration, the one or more movable talons 326 may be positioned closer to the second central axis 322 resulting in the narrower profile. The narrow profile may enable the deployable fixation element 324 and the distal body portion 320 to be advanced through a hole formed in the intramedullary canal of a fractured bone. When the distal body portion 320 is positioned within a portion of a hole (e.g., the hole 108 formed in the second bone fragment 104 of the fractured bone 100), the deployable fixation element 324 may transition to the deployed configuration.

In the deployed configuration, at least a portion of the one or more movable talons 326 moves away from the second central axis 322 thereby defining the wider profile. In the deployed configuration one or more of the movable talons 326 may directly abut an inner surface of the second bone fragment 104 (e.g., that forms a perimeter of the hole 108). Abutment of the deployable fixation element 324 with the second bone fragment 104 may restrict movement of the distal body portion 320 relative to the second bone fragment 104.

In the insertion configuration, the distal body portion 320 (e.g., the deployable fixation element 324) may define a second maximum cross-sectional dimension K2 measured in a second plane P2. The second plane P2 may be normal to the second central axis 322 and/or may be parallel to the first plane P1. The distal body portion 320 may be devoid of any transverse fixation holes, as shown in the illustrated embodiment. Alternatively, one or more transverse fixation holes may be formed in the distal body portion 320.

As used herein when referring to portions of the intramedullary implant 112, “proximal” refers to a portion (e.g., a proximal body portion) that is closer to the surgeon implanting the intramedullary implant 112 and/or closer to an incision through which the intramedullary implant 112 was inserted when fully inserted and “distal” refers to a portion (e.g., a distal body portion) that is farther from the surgeon implanting the intramedullary implant 112 and/or closer to an incision through which the intramedullary implant 112 was inserted when fully inserted. The distal/proximal of the anatomy may not match the distal/proximal of the intramedullary implant 112 (e.g., the proximal body portion 310 may be secured within a distal bone fragment and the distal body portion 320 may be secured within a proximal bone fragment).

Some embodiments of the intramedullary implant 112 may comprise an intermediate body portion 330 between the proximal body portion 310 and the distal body portion 320. The intermediate body portion 330 may extend along the first central axis 312, as shown. The intermediate body portion 330 may define a transverse fixation hole 334 that extends through the intermediate body portion 330 and intersects the first central axis 312. The intermediate body portion 330 may define a third maximum cross-sectional dimension K3 measured in a third plane P3 that is normal to the first central axis 312. The third maximum cross-sectional dimension K3 may change along a length of the intermediate body portion 330 (e.g., reducing as the intermediate body portion 330 approaches the distal body portion 320).

The transverse fixation hole 334 of the intermediate body portion 330 may be parallel with at least one of the plurality of transverse fixation holes 314 of the proximal body portion 310. For example, the transverse fixation hole 334 may be parallel to the first transverse fixation hole 315a. As shown, the first transverse fixation hole 315a may be adjacent to (and proximal of) the transverse fixation hole 334 of the intermediate body portion 330, and further be adjacent to (and distal of) the second transverse fixation hole 315b of the proximal body portion 320. Accordingly, some embodiments of the intramedullary implant 112 may comprise a set of three transverse fixation holes parallel to each other and in series along the length L of the implant body 300. The set of three transverse fixation holes may comprise the transverse fixation hole 334, the first transverse fixation hole 315a, and the second transverse fixation hole 315b. The set of three transverse fixation holes may be positioned such that first and second fragments of a fractured bone may be secured to the intramedullary implant 112 via insertion of screws that are parallel to each other on either side of a fracture location (e.g., one screw inserted into a first bone fragment and another screw inserted into a second bone fragment).

The transverse fixation hole 334 may be positioned along a length L of the intramedullary implant 112 at a location that is closer to the proximal end 302 of the implant body 300 than the location is from the distal end 304 of the implant body 300. According to some embodiments, the location of the transverse fixation hole 334 may be at least 1.5 times (e.g., at least 2 times) farther from the distal end 304 than the location is from the proximal end 302. According to some embodiments, the location of the transverse fixation hole 334 may be between about 1.5 times and about 2 times farther from the distal end 304 than the location is from the proximal end 302.

As shown in FIG. 21, each of the plurality of transverse fixation holes 314 and the transverse fixation hole 334 may extend through the implant body 300 along respective hole axes. For example, the first transverse fixation hole 315a may extend along a first hole axis 318a and the second transverse fixation hole 315b may extend along a second hole axis 318b. The first hole axis 318a and the second hole axis 318b may be parallel to one another (e.g., such that when viewed from the proximal end 302 they appear colinear). Similarly, the third transverse fixation hole 316a may extend along a third hole axis 318c and the fourth transverse fixation hole 316b may extend along a fourth hole axis 318d. The third hole axis 318c and the fourth hole axis 318d may be parallel to one another (e.g., such that when viewed from the proximal end 302 they appear colinear). Similarly, the fifth transverse fixation hole 317a may extend along a fifth hole axis 318e and the sixth transverse fixation hole 317b may extend along a sixth hole axis 318f. The fifth hole axis 318e and the sixth hole axis 318f may be parallel to one another (e.g., such that when viewed from the proximal end 302 they appear colinear).

The transverse fixation hole 334 of the intermediate body portion 330 may extend along a hole axis 338. As shown in FIG. 24, the hole axis 338 may be parallel to the first hole axis 318a and the second hole axis 318b (e.g., such that when viewed from the proximal end 302 they appear colinear). The hole axis 338 may be angularly offset from the third hole axis 318c and the fourth hole axis 318d by an angle α. According to some embodiments, the angle α may range from 0 degrees to 90 degrees, e.g., from 5 degrees to 70 degrees, from 10 degrees to 50 degrees, from 15 degrees to 40 degrees, or from 20 degrees to 30 degrees. In terms of lower limits, the angle α may be greater than 5 degrees, e.g., greater than 10 degrees, greater than 15 degrees, greater than 20 degrees, greater than 30 degrees, greater than 40 degrees, greater than 50 degrees, or greater than 70 degrees. In terms of upper limits, the angle α may be less than 90 degrees, e.g., less than 70 degrees, less than 50 degrees, less than 40 degrees, less than 30 degrees, less than 20 degrees, less than 15 degrees, less than 10 degrees, or less than 5 degrees. For example, the angle α may be about 25 degrees (e.g., plus or minus 5 degrees).

The hole axis 338 may be angularly offset from the fifth hole axis 318e and the sixth hole axis 318f by an angle β. According to some embodiments, the angle β may range from 0 degrees to 90 degrees, e.g., from 10 degrees to 85 degrees, from 20 degrees to 80 degrees, from 30 degrees to 75 degrees, or from 50 degrees to 70 degrees. In terms of lower limits, the angle β may be greater than 10 degrees, e.g., greater than 20 degrees, greater than 30 degrees, greater than 40 degrees, greater than 50 degrees, greater than 70 degrees, greater than 75 degrees, greater than 80 degrees, or greater than 85 degrees. In terms of upper limits, the angle β may be less than 90 degrees, e.g., less than 85 degrees, less than 80 degrees, less than 75 degrees, less than 70 degrees, less than 50 degrees, less than 40 degrees, less than 30 degrees, less than 20 degrees, or less than 10 degrees. For example, the angle β may be about 60 degrees (e.g., plus or minus 10 degrees).

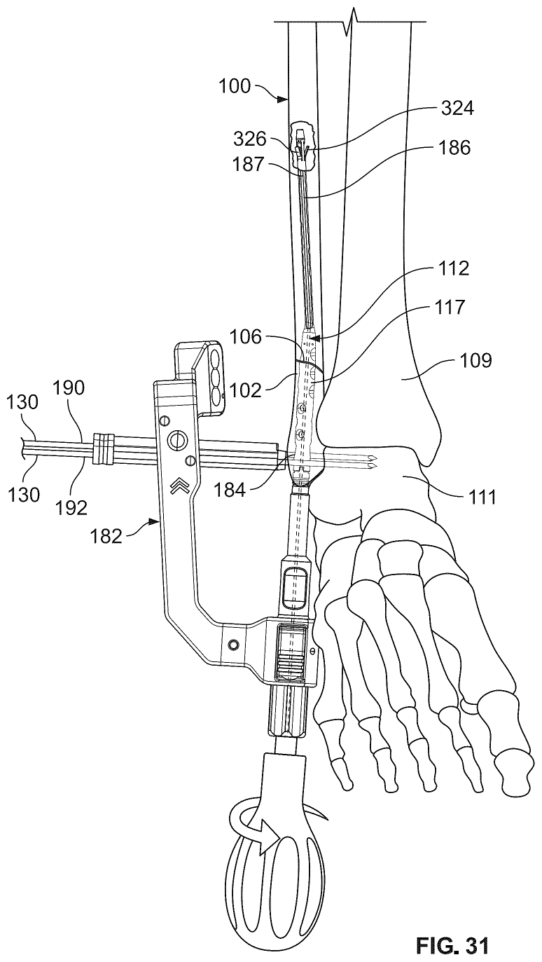

Some embodiments of the intramedullary implant 112 may comprise an axial bore 340 that extends through the proximal body portion 310, along the first central axis 312, through the intermediate body portion 330, and into the distal body portion 320 (e.g., along at least a portion of the second central axis 322). The axial bore 340 may intersect the transverse fixation hole 334 of the intermediate body portion 330 and any, up to all, of the plurality of transverse fixation holes 314 of the proximal body portion 310. The intramedullary implant 112 may comprise an actuator (e.g., the actuator 186 shown in FIG. 31) with a shaft that is sized to advance along the axial bore 340 and engage the deployable fixation element 324. The actuator may further comprise a handle accessible outside of the patient's body that can be rotated to rotate the shaft to thereby transition the deployable fixation element 324 from the insertion configuration to the deployed configuration.

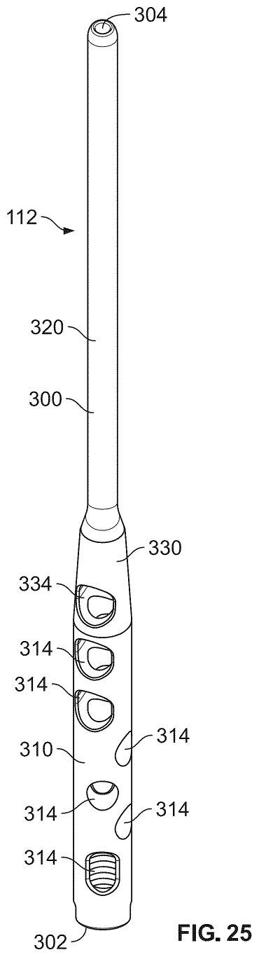

As shown in FIG. 25, some embodiments of the intramedullary implant 112 may be devoid of the deployable fixation element 324, but otherwise identical to other embodiments of the intramedullary implant 112 as described herein. Accordingly, some embodiments of the intramedullary implant 112 may be securable to the second bone fragment 104 without the use of an internal/deployable fixation element, and may instead be secured via a bone screw inserted through the transverse fixation hole 334 of the intermediate body portion 330.

Referring to FIGS. 13 to 17, a method of assembling the bone fracture repair kit 400 may comprise assembling the coupler 500 by inserting the fastener distal end 542 into and through the sleeve lumen 516. The method may further comprise connecting the fastener distal end 542 to the intramedullary implant 112 while a portion of the fastener 502 is within the sleeve lumen 516. According to the method the actuator 414 of the targeting guide 182 may abut the engagement portion 524 of the sleeve 504 to secure a position of the sleeve 504 relative to the targeting guide 182. The actuator 414 may be disengaged from the engagement portion 524 of the sleeve 504 and then the targeting guide 182 may be moved relative to the sleeve 504, the fastener 502, and intramedullary implant 112 while the actuator 414 is disengaged from the engagement portion 524.

According to some embodiments, disengaging the actuator 414 from the engagement portion 524 of the sleeve 504 may comprise pivoting a spring biased lever about a pivot axis (e.g., to remove a portion of the lever from the recess 526). Disengaging the targeting guide 182 from the coupler 500 may comprise removing multiple actuators 414 (e.g., first and second spring biased levers) from respective recesses 526. For example, two actuators 414 may need to be pressed at the same time to remove multiple spring biased levers from multiple recesses 526 to enable movement of the targeting guide 182 relative to the coupler 500.

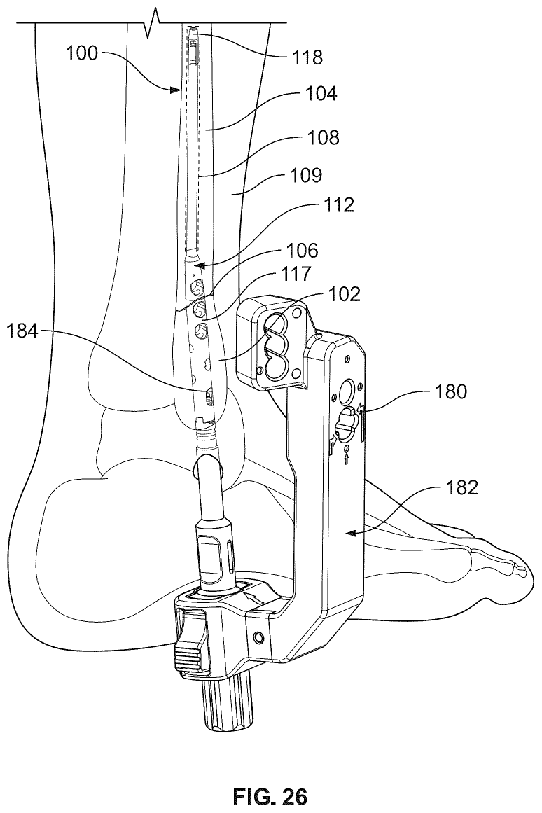

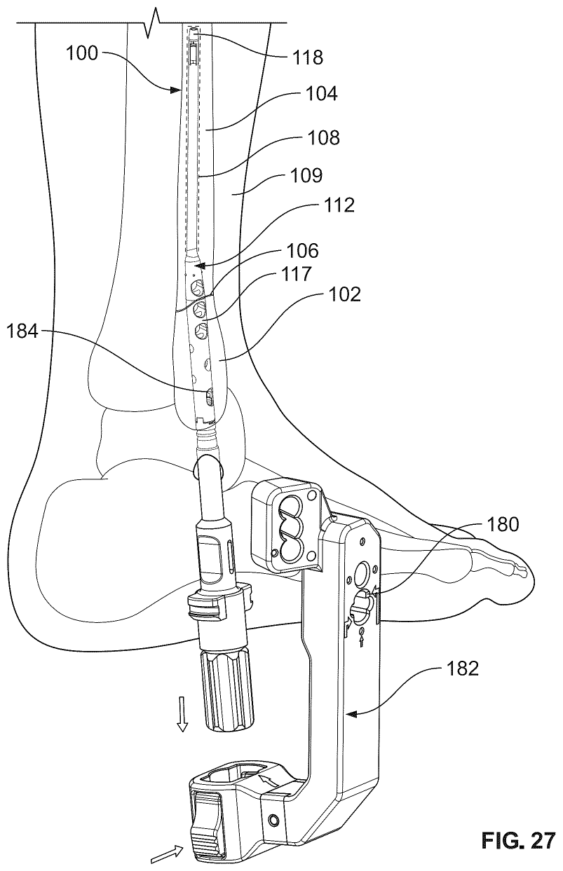

Referring to FIGS. 1 to 35, a method of fixating a first bone fragment (e.g., the first bone fragment 102) relative to a second bone fragment (e.g., the second bone fragment 104) may comprise advancing the distal body portion 320 of the intramedullary implant 112 through an opening formed in the first bone fragment 102 and through a hole (e.g., the hole 108, a medullary cavity, etc.) defined by the first bone fragment 102. The method may comprise moving the distal body portion 320 across the fracture 106 between the first bone fragment 102 and the second bone fragment 104 and advancing the distal body portion 320 into and through a portion of a medullary cavity of the second bone fragment 104.

Some embodiments of the method may further comprise advancing the proximal body portion 310 of the intramedullary implant 112 through the opening formed in the first bone fragment 102 and advancing the proximal body portion 310 into the medullary cavity of the first bone fragment 102. When the distal body portion 320 is positioned within the second bone fragment 104 the deployable fixation element 324 may be transitioned from the insertion configuration to the deployed configuration in which the movable talons 326 contact an inner wall of the second bone fragment 104. After transitioning the deployable fixation element 324, a first bone fixation element (e.g., a bone screw 350) may be inserted through an exterior surface of the second bone fragment 104 and through the transverse fixation hole 334 of the intramedullary implant 112. According to the method, a second bone fixation element (e.g., a bone fixator 194 such as a bone screw) may be inserted through an exterior surface of the first bone fragment 102 and through one of the plurality of transverse fixation holes 314 of the proximal body portion 310 of the intramedullary implant 112.

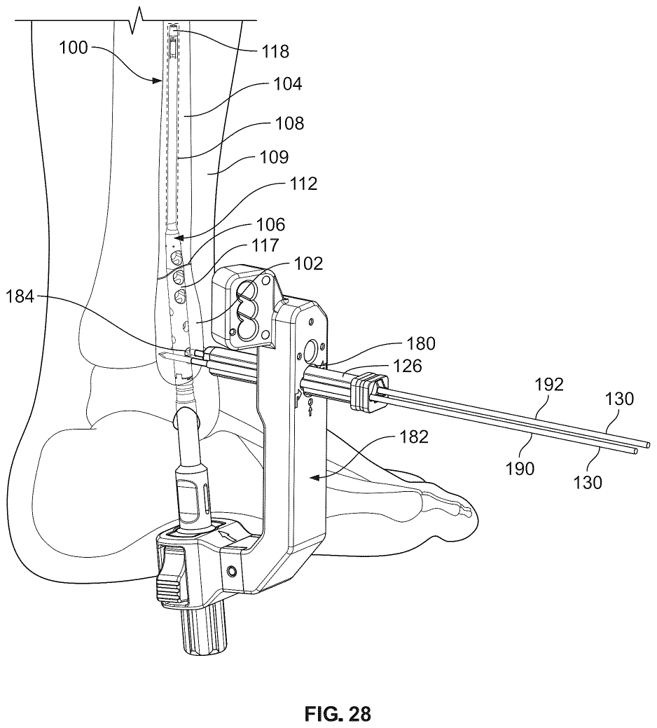

As shown in FIGS. 28 to 30, the method may comprise inserting a multi-lumen cannula 120 through a recess 180 of a targeting guide 182 that is secured to the intramedullary nail 117. The recess 180 may have a “keyed” shape that corresponds to an outer perimeter of a body 126 of the multi-lumen cannula 120. Thus, when positioned within the recess 180, the multi-lumen cannula 120 may not be rotatable relative to the targeting guide 182. The method may further include advancing the multi-lumen cannula 120 toward the intramedullary nail 117 (e.g., until a gap between the multi-lumen cannula 120 and the intramedullary nail 117 is minimized, for example below about 20 mm).

As shown in FIG. 28, the method may further include advancing a first bone fixator 190 (e.g., a K-wire) through a first lumen of the multi-lumen cannula (e.g., along a first lumen axis and advancing a second bone fixator 192 (e.g., a K-wire) through a second lumen (e.g., along a second lumen axis). When the multi-lumen cannula 120 is positioned within the recess 180 and the targeting guide 182 is secured to the intramedullary nail 117, the first bone fixator 190 may be advanced along the first lumen axis passing on one side of the intramedullary nail 117, and the second bone fixator 192 may be advanced along the second lumen axis passing on the other side of the intramedullary nail 117.

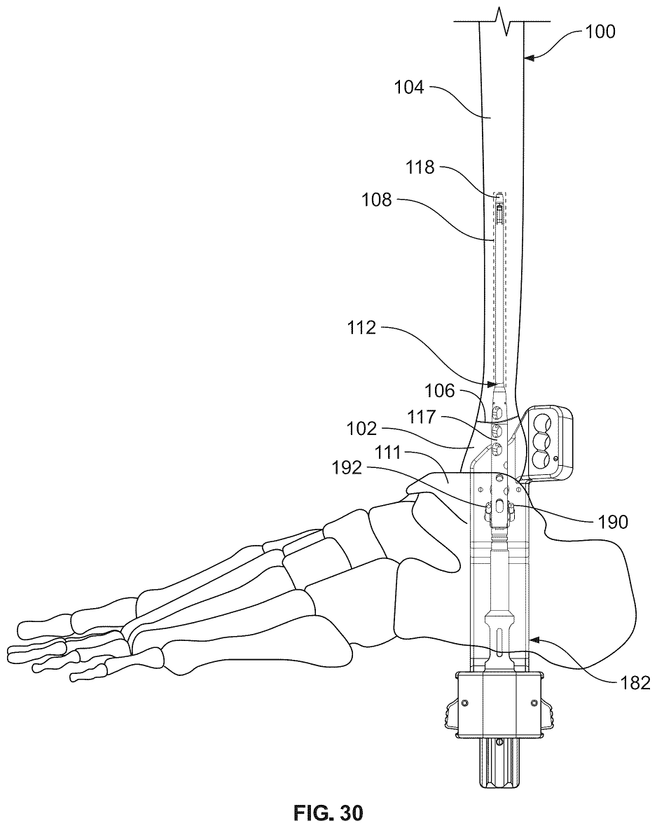

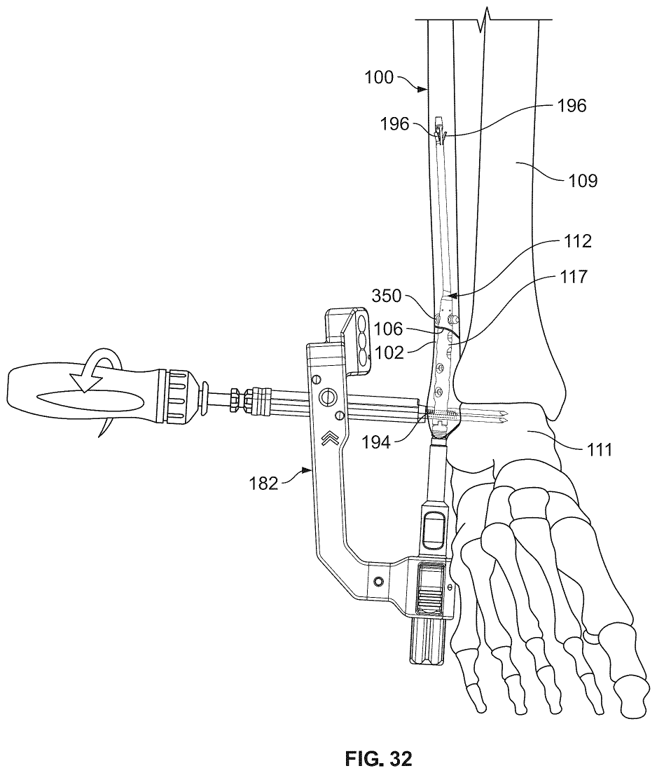

As shown in FIG. 30, the method may include bracketing the intramedullary nail 117 between the first bone fixator 190 and the second bone fixator 192. As shown in FIG. 32, the method may further include advancing a third bone fixator 194 (e.g., a bone screw) through a third lumen of the multi-lumen cannula 120 (e.g., along a third lumen axis). When the multi-lumen cannula 120 is positioned within the recess 180 and the targeting guide 182 is secured to the intramedullary nail 117, the third bone fixator 194 may be advanced along the third lumen axis until passing through a transverse fixation hole 314 of the intramedullary nail 117. According to some embodiments, the third lumen axis and the intramedullary nail 117 may be substantially perpendicular to one another (e.g., within about 10 degrees of offset). Thus, a central axis of the transverse fixation hole 314 may be perpendicular to the third lumen axis, or offset from the third lumen axis by up to about 10 degrees.

The multi-lumen cannula 120 may be in a first orientation relative to the targeting guide 182 when the multi-lumen cannula 120 is positioned within the recess 180. The method may further comprise withdrawing the multi-lumen cannula 120 through the recess 180 of the targeting guide 182, rotating the multi-lumen cannula 120 (e.g., 180 degrees) to a second orientation, and then inserting the multi-lumen cannula through the recess 180 of the targeting guide 182 while the multi-lumen cannula 120 is in the second orientation. When the multi-lumen cannula 120 is positioned within the recess 180 in the second orientation, the first bone fixator 190, the second bone fixator 192, and/or the third bone fixator 194 may be inserted through the first lumen, the second lumen, and/or the third lumen, respectively, as described above in reference to the first orientation.

As shown in FIG. 31, the method may include advancing an actuator 186 along a length of the intramedullary nail 117, passing the actuator 186 between the first bone fixator 190 and the second bone fixator 192. According to some embodiments of the method, the actuator 186 may be advanced prior to advancing the third bone fixator 194 into the bone fastener receiving hole 184 of the intramedullary nail 117. The actuator 186 may be advanced through a channel within the intramedullary nail 117, and the channel may be blocked by the third bone fixator 194, when the third bone fixator 194 is positioned within the bone fastener receiving hole 184.

The method may include advancing an actuator 186 within the intramedullary nail 117 until the actuator 186 engages the deployable fixation element 324 of the intramedullary nail 117. The actuator 186 may be rotated within the intramedullary nail 117 while the actuator 186 is engaged with the deployable bone fixator 196 to deploy the deployable bone fixator 196 (e.g., such that the deployable bone fixator 196 engages with an inner surface of the second bone fragment 104). Once the deployable fixation element 324 is in the deployed configuration, the method may include withdrawing the actuator 186 from the intramedullary nail 117 prior to advancing the third bone fixator 194 into the transverse fixation hole 314.

According to some embodiments, a method of repairing a fractured bone (e.g., the fractured bone 100) may include positioning the intramedullary nail 117 in an intramedullary canal 110 (e.g., the hole 108) of the fractured bone 100 such that a portion of the intramedullary nail 117 is in the first bone fragment 102 of the fractured bone 100, another portion of the intramedullary nail 117 is in the second bone fragment 104 of the fractured bone 100, and the intramedullary nail 117 extends across the fracture 106 between the first bone fragment 102 and the second bone fragment 104.

For example, when inserting the multi-lumen cannula 120 through the recess 180 of the targeting guide 182, the targeting guide 182 may be secured to the intramedullary nail 117, which may be positioned within the hole 108 formed in the fractured bone 100. The multi-lumen cannula 120 may be advanced toward the intramedullary nail 117, according to the method, until the multi-lumen cannula 120 contacts the skin of the patient or the fractured bone 100.

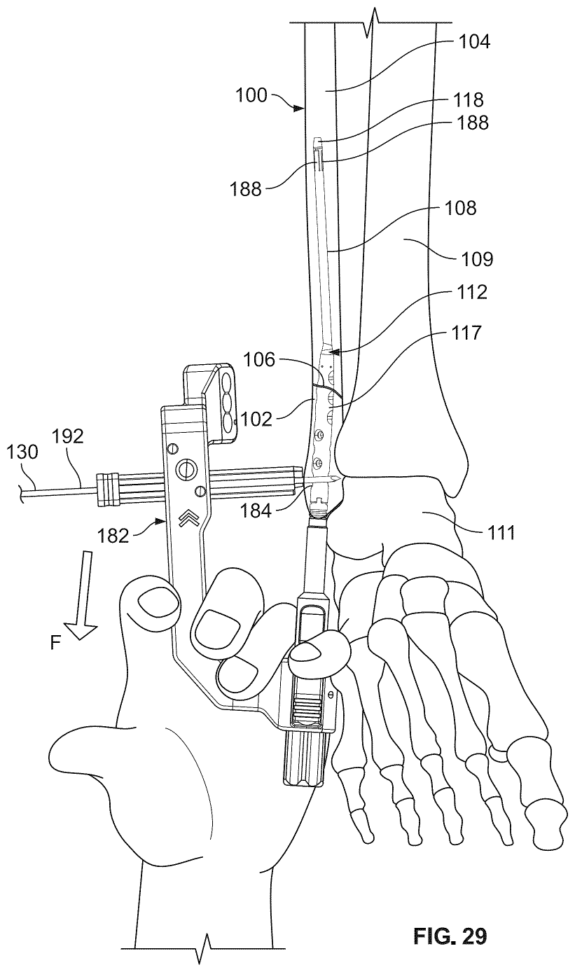

When the intramedullary nail 117 is positioned within the hole 108 formed in the fractured bone 100, the method may include advancing the first bone fixator 190 into the first bone fragment 102 (e.g., a distal fragment of a fibula) and advancing the second bone fixator 192 into the second bone fragment 104. As shown in FIG. 29, a force F may be applied to the multi-lumen cannula 120, the targeting guide 182, the first bone fixator 190, the second bone fixator 192, or any combination thereof. When the first bone fixator 190 is inserted into and secured to the first bone fragment 102 (e.g., a distal fragment of a fibula) and the second bone fixator 192 is inserted into and secured to the second bone fragment 104, the force F moves the first bone fragment 102 away from the second bone fragment 104.

Thus, the method may include increasing a length of the fractured bone 100 with the multi-lumen cannula 120 used as a fracture reduction sleeve. The method may include rotating the first bone fragment 102 relative to the second bone fragment 104 of the fractured bone 100 (e.g., to realign the first bone fragment 102 and the second bone fragment 104 to match the pre-fractured condition of the fractured bone 100). However, the multi-lumen cannula 120 may be used in other procedures involving the insertion of bone fixators along desired pathways. Implantation of the intramedullary nail 117 and methods of bone fixation described herein may be performed without the use of the multi-lumen cannula 120. For example, temporary fixators (e.g., K-wires) may be inserted into the first bone fragment 102 and/or the second bone fragment 104 (either with or without the use of the targeting guide 182 and with or without the use of the multi-lumen cannula 120). When inserted, force may be applied to the temporary fixators to manipulate the first bone fragment 102 relative to the second bone fragment 104.

When the intramedullary nail 117 is positioned within the hole 108 formed in the fractured bone 100, the method may include securing the intramedullary nail 117 to the first bone fragment 102 via insertion of the third bone fixator 194. Transitioning the deployable bone fixator 196 may include abutting the movable talons 326 with the inner surface of the second bone fragment 104 of the fractured bone 100. Advancing the actuator 186 along the length of the intramedullary nail 117 may include moving a tip 187 of the actuator 186 across the fracture 106 and passing the tip 187 between the first bone fixator 190 and the second bone fixator 192.

As shown in FIG. 29, when the fractured bone 100 has the desired length (e.g., while the force F is being applied to the first bone fragment 102 (e.g., via the multi-lumen cannula 120, the targeting guide 182, the first bone fixator 190, the second bone fixator 192, or any combination thereof) the first bone fixator 190 and the second bone fixator 192 may be advanced further (e.g., medially into the body) to enter a second bone (e.g., a tibia 109 and/or a talus 111). Thus the method may include securing a position of the first bone fragment 102 to another bone (e.g., not the fractured bone 100 or any of its fragments). When the first bone fragment 102 is secured to the other bone, the force F may be removed as it is no longer needed to maintain the desired length of the fractured bone 100, and the position of the first bone fragment 102 relative to the second bone fragment 104.

As shown in FIG. 31, rotating the actuator 186 within the intramedullary nail 117 may deploy the one or more talons 188 thereby securing the intramedullary nail 117 to the second bone fragment 104. After deploying the one or more talons 188, the actuator 186 may be withdrawn entirely from the intramedullary canal 110 of the intramedullary nail 117 prior to advancing the third bone fixator 194 into the bone fastener receiving hole 184, as shown in FIG. 32. Inserting the third bone fixator 194, according to the method, may secure the intramedullary nail 117 to the first bone fragment 102. The bone screws inserted through the transverse fixation holes 314, 334 of the intramedullary nail 117 may be inserted in different orders. For example, the bone screw 350 may be inserted through the transverse fixation hole 334 of the intermediate body portion 330 prior to insertion of any bone screws being inserted through the transverse fixation holes 314 of the proximal body portion 310. Alternatively, the bone screw 350 may be inserted through the transverse fixation hole 334 of the intermediate body portion 330 after insertion of any/all bone screws being inserted through the transverse fixation holes 314 of the proximal body portion 310.

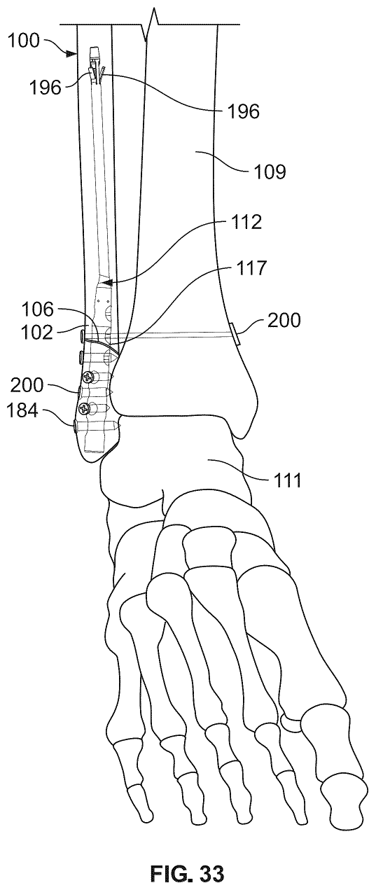

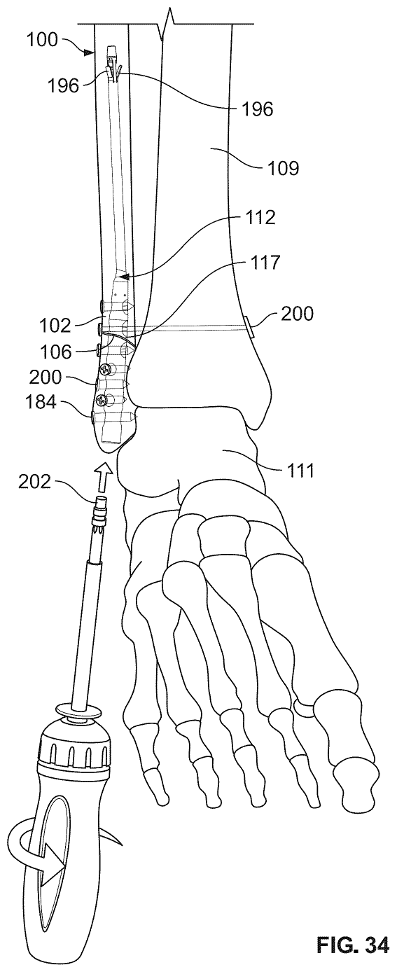

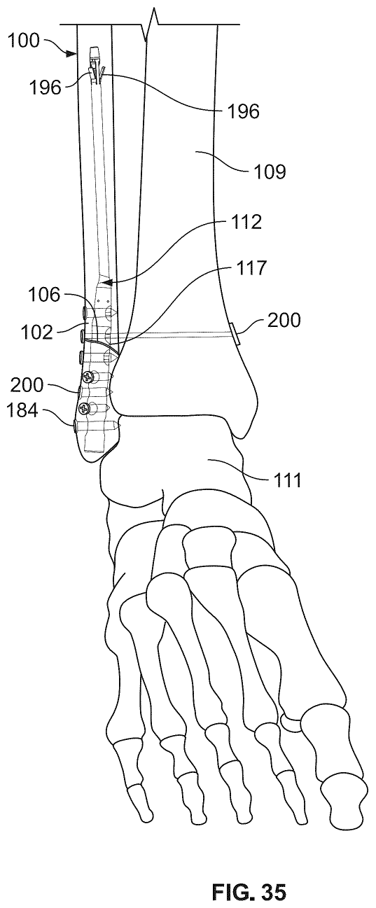

As shown in FIG. 33, additional permanent bone fixators 200 (e.g., bone screws similar to the bone screw 350) may be inserted into respective ones of the plurality of transverse fixation holes 314 to secure the proximal body portion 310 of the intramedullary implant 112 to the first bone fragment 102 of the fractured bone 100. According to some embodiments, all of the additional permanent bone fixators 200 may be inserted into corresponding bone fastener receiving holes to secure the intramedullary implant 112 to the first bone fragment 102 of the fractured bone 100. As shown in FIG. 34, after insertion of the additional permanent bone fixators 200 (e.g., when the intramedullary nail 117 is fully secured to the fractured bone 100), an end cap 202 may be coupled to the intramedullary nail 117 to close the path through which the actuator 186 passed. As shown in FIG. 35, the completed intramedullary nail 117 and the permanent bone fixators remain secured to the fractured bone 100, and the temporary bone fixators may be removed.

Although described above in use to fixate first and second fragments of a fractured bone, the intramedullary implant 112 and its methods of use are not so limited. The fractured bone 100 may comprise more than two (e.g., three, four, five, etc.) bone fragments that need to be repositioned and secured to restore the fractured bone 100 to its pre-fracture condition (e.g., a butterfly fracture).

According to some embodiments, the deployable fixation element 324 may be secured within one bone fragment, one or more of the plurality of transverse fixation holes 314 may be secured within another bone fragment, and the transverse fixation hole 334 of the intermediate body portion 330 may be secured within a third bone fragment. For example, a method of fixating multiple fragments of a fractured bone relative to one another may comprise advancing the distal body portion 320 through an opening formed in a first bone fragment of the fractured bone 100. The method may further comprise advancing the distal body portion 320 through a medullary cavity of the first bone fragment and advancing the distal body portion 320 into and through a portion of a medullary cavity of a second bone fragment of the fractured bone 100.

The method may further comprise advancing the proximal body portion 310 of the intramedullary implant 112 through the opening formed in the first bone fragment and advancing the proximal body portion 310 into the medullary cavity of the first bone fragment. The method may comprise deploying the deployable fixation element 324 of the distal body portion 320 within the medullary cavity of the second bone fragment until the fixation element contacts an inner wall of the second bone fragment that defines the medullary cavity of the second bone fragment. A first bone fixation element may be inserted through an exterior surface of a third bone fragment of the fractured bone 100 that is between the first and second bone fragments of the fractured bone 100 and through the transverse fixation hole 334 defined by the intermediate body portion 330 of the intramedullary implant 112. A second bone fixation element may be inserted through an exterior surface of the first bone fragment and through one of the plurality of transverse fixation holes 314 defined by the proximal body portion 310 of the intramedullary implant 112.

The above description of illustrated embodiments, including what is described in the Abstract, is not intended to be exhaustive or to limit the embodiments to the precise forms disclosed. Although specific embodiments of and examples are described herein for illustrative purposes, various equivalent modifications can be made without departing from the spirit and scope of the disclosure, as will be recognized by those skilled in the relevant art. The various embodiments described above can be combined to provide further embodiments.

Many of the methods described herein can be performed with variations. For example, many of the methods may include additional acts, omit some acts, and/or perform acts in a different order than as illustrated or described.

As used herein, “greater than” and “less than” limits may also include the number associated therewith. Stated another way, “greater than” and “less than” may be interpreted as “greater than or equal to” and “less than or equal to.” It is contemplated that this language may be subsequently modified in the claims to include “or equal to.” For example, “greater than 2 mm” may be interpreted as, and subsequently modified in the claims as “greater than or equal to 2 mm.”

In some embodiments, any or some of the components or steps disclosed herein may be considered optional. In some cases, the disclosed embodiments may expressly exclude any or some of the aforementioned elements or steps in this description, e.g., via claim language. For example, claim language may be modified to recite that the disclosed intramedullary implant and/or methods, etc., do not utilize or comprise a deployable fixation element or a multi-lumen cannula. Such negative limitations are contemplated, and this text serves as support for negative limitations for components, steps, and/or features.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1. A coupler that releasably attaches an intramedullary implant to a targeting guide used to secure the intramedullary implant to one or more fragments of a fractured bone via fasteners inserted through corresponding holes of the intramedullary implant and the targeting guide that are aligned when the intramedullary implant is attached to the targeting guide by the coupler, the coupler comprising:
a sleeve comprising:
a sleeve proximal end, a sleeve distal end, a sleeve body extending therebetween, and defining a sleeve lumen extending therethrough along a first longitudinal axis,
wherein the sleeve comprises an engagement portion that is shaped to releasably engage the targeting guide such that translation of the sleeve relative to the targeting guide along the first longitudinal axis is restricted; and
a fastener comprising:
a fastener proximal end, a fastener distal end, a fastener body extending therebetween, and defining a fastener lumen extending therethrough along a second longitudinal axis,
wherein the fastener distal end is shaped to engage the intramedullary implant such that translation of the fastener relative to the intramedullary implant along the second longitudinal axis is restricted, and
wherein at least a portion of the fastener body is shaped to be inserted into and translated through the sleeve lumen.

2. The coupler of claim 1 wherein the sleeve distal end is shaped to engage the intramedullary implant such that rotation of the sleeve relative to the intramedullary implant about the first longitudinal axis is restricted.

3. The coupler of claim 2 wherein the sleeve distal end comprises a flange extending distally from a remainder of the sleeve body, wherein the flange defines a radial length that is less than a full circumference around the first longitudinal axis.

4. The coupler of claim 3 wherein the flange is a first flange and the sleeve distal end comprises a second flange extending distally from the remainder of the sleeve body.

5. The coupler of claim 4 wherein the second flange has a radial length equal to the radial length of the first flange.

6. The coupler of claim 3 wherein the radial length of the flange ranges from 10 to 90 degrees.

7. The coupler of claim 1 wherein the engagement portion of the sleeve defines a recess that extends radially toward the first longitudinal axis along a length of the engagement portion measured parallel to the first longitudinal axis.

8. The coupler of claim 7 wherein the engagement portion of the sleeve comprises a tapered surface that extends radially away from the first longitudinal axis as the engagement portion extends distally.

9. The coupler of claim 8 wherein the tapered surface is positioned proximally of the recess.

10. The coupler of claim 8 wherein the recess is a first recess, the tapered surface is a first tapered surface, and the engagement portion of the sleeve comprises:
a second recess that extends radially toward the first longitudinal axis along the length of the engagement portion; and
a second tapered surface that extends radially away from the first longitudinal axis as the engagement portion extends distally.

11. The coupler of claim 10 wherein the first recess and the second recess are positioned such that a first plane that is normal to the first longitudinal axis intersects both the first recess and the second recess.

12. The coupler of claim 10 wherein the first tapered surface and the second tapered surface are positioned such that a second plane that is normal to the first longitudinal axis intersects both the first tapered surface and the second tapered surface.

13. The coupler of claim 1 wherein the fastener distal end comprises a threaded surface.

14. The coupler of claim 13 wherein the threaded surface of the fastener distal end comprises external threads that correspond to internal threads of the intramedullary implant.

15. The coupler of claim 13 wherein the fastener is positionable within the sleeve lumen such that at least a portion of the threaded surface of the fastener distal end extends distally beyond sleeve distal end.

16. The coupler of claim 1, further comprising:
an engagement portion of the fastener that is shaped to engage the sleeve such that translation of the fastener relative to the sleeve along the first longitudinal axis is restricted.

17. The coupler of claim 16 wherein the engagement portion of the fastener comprises a threaded surface.

18. The coupler of claim 17 wherein the threaded surface of the engagement portion comprises external threads that correspond to internal threads of the sleeve.

19. The coupler of claim 1 wherein the fastener comprises a stop that abuts the sleeve to prevent movement of the fastener distally relative to the sleeve.

20. The coupler of claim 19 wherein the stop abuts the sleeve proximal end.

21. A coupler that releasably attaches an intramedullary implant to a targeting guide used to secure the intramedullary implant to one or more fragments of a fractured bone via fasteners inserted through corresponding holes of the intramedullary implant and the targeting guide that are aligned when the intramedullary implant is attached to the targeting guide by the coupler, the coupler comprising:
a sleeve comprising:
a sleeve proximal end, a sleeve distal end, a sleeve body extending therebetween, and defining a sleeve lumen extending therethrough along a first longitudinal axis,
wherein the sleeve comprises an engagement portion defining a recess that extends into the sleeve body radially toward the first longitudinal axis along a length of the engagement portion; and
a fastener comprising:
a fastener proximal end, a fastener distal end, a fastener body extending therebetween, and defining a fastener lumen extending therethrough along a second longitudinal axis, wherein the fastener distal end comprises a threaded surface, and
herein a cross-sectional shape of a distal portion of the fastener body fits within the sleeve lumen.

22. The coupler of claim 21 wherein the sleeve distal end comprises one or more flanges extending distally from a remainder of the sleeve body, and wherein the one or more flanges each define a radial length that is less than a full circumference around the first longitudinal axis.

23. The coupler of claim 22 wherein the one or more flanges includes a first flange and a second flange separated by a radial gap.

24. The coupler of claim 23 wherein the radial length of the first flange is equal to the radial length of the second flange.

25. The coupler of any one of claims 22 to 24 claim 22 wherein the radial length of each of the one or more flanges ranges from 10 to 90 degrees.

26. The coupler of claim 21 wherein the engagement portion of the sleeve comprises a tapered surface that extends radially away from the first longitudinal axis as the engagement portion extends distally.

27. The coupler of claim 26 wherein the tapered surface is positioned proximally of the recess.

28. The coupler of claim 26 wherein the recess is a first recess, the tapered surface is a first tapered surface, and the engagement portion of the sleeve comprises:
a second recess that extends radially toward the first longitudinal axis along the length of the engagement portion; and
a second tapered surface that extends radially away from the first longitudinal axis as the engagement portion extends distally.

29. The coupler of claim 28 wherein the first recess and the second recess are positioned such that a first plane that is normal to the first longitudinal axis intersects both the first recess and the second recess.

30. The coupler of claim 28 wherein the first tapered surface and the second tapered surface are positioned such that a second plane that is normal to the first longitudinal axis intersects both the first tapered surface and the second tapered surface.

31. The coupler of claim 21 wherein the threaded surface of the fastener distal end comprises external threads that correspond to internal threads of the intramedullary implant.

32. The coupler of claim 31 wherein the fastener is positionable within the sleeve lumen such that at least a portion of the threaded surface of the fastener distal end extends distally beyond sleeve distal end.

33. The coupler of claim 21, further comprising:
corresponding threads on an outer surface of the fastener and an inner surface of the sleeve, wherein the corresponding threads threadedly engage such that rotation of the fastener relative to the sleeve moves the fastener along the first longitudinal axis relative to the sleeve.

34. The coupler of claim 21 wherein the fastener comprises a stop that abuts the sleeve to prevent movement of the fastener distally relative to the sleeve.

35. A bone fracture repair kit comprising:
an intramedullary implant having a bone fixation hole that receives a fastener to secure the intramedullary implant to a bone;
a targeting guide having a fastener guide hole; and
the coupler of claim 1;
wherein that bone fracture repair kit defines an assembled configuration in which an actuator of the targeting guide engages an engagement portion of the sleeve thereby securing the intramedullary implant to the targeting guide such that movement of the intramedullary implant relative to the targeting guide is restricted and such that the bone fixation hole is aligned with the fastener guide hole, and
wherein the bone fracture repair kit defines a released configuration in which the actuator is removed from engagement with the engagement portion of the targeting guide thereby releasing the intramedullary implant from the targeting guide such that the targeting guide is movable relative to the intramedullary implant.

36. The bone fracture repair kit of claim 35 wherein the sleeve distal end and a proximal end of the intramedullary implant have complementary surfaces that engage such that rotation of the sleeve relative to the intramedullary implant about the first longitudinal axis is restricted.

37. The bone fracture repair kit of claim 36 wherein the proximal end of the intramedullary implant comprises one or more recesses extending distally into a body of the intramedullary implant, wherein the one or more recess each define a radial length that is less than a full circumference around the first longitudinal axis.

38. The bone fracture repair kit of claim 35 wherein the actuator comprises a spring biased lever that abuts the engagement portion of the sleeve to restrict translation of the sleeve relative to the targeting guide.

39. The bone fracture repair kit of claim 35 wherein the targeting guide defines a through hole that receives the sleeve therethrough.

40. The bone fracture repair kit of claim 39 wherein the through hole of the targeting guide and an outer surface of the sleeve have corresponding non-circular shapes that restrict rotation of the sleeve relative to the targeting guide when the sleeve is received within the through hole of the targeting guide.

41. A method of assembling a bone fracture repair kit, the method comprising:
assembling a coupler comprising a fastener and a sleeve, wherein the sleeve has sleeve proximal end, a sleeve distal end, a sleeve body extending therebetween, and a sleeve lumen extending therethrough along a first longitudinal axis, and wherein the fastener has a fastener proximal end, a fastener distal end, a fastener body extending therebetween, and defining a fastener lumen extending therethrough along a second longitudinal axis;
inserting the fastener distal end into and through the sleeve lumen;
connecting the fastener distal end to an intramedullary implant while a portion of the fastener is within the sleeve lumen;
abutting an actuator of a targeting guide with an engagement portion of the sleeve to secure a position of the sleeve relative to the targeting guide;
disengaging the actuator from the engagement portion of the sleeve; and
moving the targeting guide relative to the sleeve, fastener, and intramedullary implant while the actuator is disengaged from the engagement portion.

42. The method of claim 41 wherein the actuator comprises a spring biased lever, the method further comprising:
pivoting the spring biased lever about a pivot axis to disengage the actuator from the engagement portion of the sleeve.

43. The method of claim 42 wherein the engagement portion comprises a recess, the method further comprising:
pivoting the spring biased lever about the pivot axis to remove the actuator from the recess.

44. The method of claim 43 wherein the spring biased lever is a first spring biased lever and the pivot axis is a first pivot axis, the method further comprising:
pivoting a second spring biased lever about a second pivot axis to disengage the actuator from the engagement portion of the sleeve.

45. The method of claim 44 wherein the recess is a first recess, the engagement portion comprises a second recess, and the method further comprises:
pivoting the second spring biased lever about the second pivot axis to remove the second spring biased lever from the second recess.

46. The method of claim 41 wherein moving the targeting guide relative to the sleeve comprises translating the targeting guide parallel to the first longitudinal axis.

47. The method of claim 41, further comprising:
sliding the actuator along a tapered surface of the engagement portion, wherein the tapered surface extends radially away from the first longitudinal axis as the engagement portion extends distally.

48. The method of claim 41, further comprising:
rotating the fastener about the second longitudinal axis relative to the sleeve to engage threads of the fastener with corresponding threads of the intramedullary implant.

49. The method of claim 41, further comprising:
rotating the fastener about the second longitudinal axis relative to the sleeve to engage threads of the fastener with corresponding threads of the sleeve.

50. The method of claim 41, further comprising:
securing an entirety of the sleeve body between a stop of the fastener and the intramedullary implant.

51. A targeting guide kit comprising:
a first body defining a first set of one or more fastener guide holes extending therethrough;
a second body defining a second set of one or more fastener guide holes extending therethrough; and
an extension defining a third set of one or more fastener guide holes extending therethrough,
wherein the extension is attachable to the first body in a first orientation, releasable from the first body, and attachable to the second body in a second orientation that is different from the first orientation.

52. The targeting guide kit of claim 51 wherein the extension is flipped vertically to transition from the first orientation to the second orientation.

53. The targeting guide kit of claim 51 wherein the first set of one or more fastener guide holes are positioned to guide fasteners to secure an intramedullary implant to a left fibula, and the second set of one or more fastener guide holes are positioned to guide fasteners to secure an intramedullary implant to a right fibula.

54. A targeting guide comprising:
an actuator that releasably attaches the targeting guide to a coupler secured to an intramedullary implant, the actuator comprising a spring biased lever that is biased into a locked configuration in which the spring biased lever is captured within a recess of the coupler,
wherein the actuator is movable to a released configuration in which the spring biased lever is removed from the recess and the targeting guide is movable relative to the coupler.

55. The targeting guide of claim 54 wherein the spring biased lever is a first spring biased lever, and the actuator comprises a second spring biased lever.

56. The targeting guide of wherein the actuator is positioned within a through hole that extends through a boom arm of the targeting guide, and wherein the through hole has a non-circular cross-sectional shape that corresponds to the coupler so as to restrict rotation of the coupler relative to the targeting guide.
